SEQUENCE LISTING (2 sequences)

SEQ ID NO: 1 - protein, 188 aa, Saccharomyces cerevisiae
MGKTADNHGPVRSETAREGRENQVYSPVTGARLVAGCICLTPDKKQVLMITSSAHKKRWIVPKGGVEKDEPNYETTAQRETWEEAGCIGKIVANLGTVEDMRPPKDWNKDIKQFENSRKDSEVAKHPPRTEFHFYELEIENLLDKFPECHKRHRKLYSYTEAKQNLIDAKRPELLEALNRSAIIKDDK

SEQ ID NO: 2 - DNA, 567 bp, Saccharomyces cerevisiae
atgggcaaaaccgcggataatcatggtccagtacgttctgagacagcacgtgaaggaagagaaaaccaggtttactcacccgttacaggtgcaagattagttgctggctgcatatgtttaacacccgacaagaagcaagttctcatgattacttcttctgcacacaagaaaagatggattgtccccaaaggtggcgttgagaaagacgagcctaattacgagacgactgcccaacgagaaacttgggaggaagctggttgcataggtaaaattgtcgccaatttgggtacagttgaagacatgagaccccctaaggactggaacaaagacattaagcaattcgagaactctcgaaaagattcagaagtagcaaagcacccgccaagaaccgaatttcatttttatgaattagagattgaaaatctccttgataaatttccggaatgtcacaaaagacatagaaagctatactcttatacagaagctaaacaaaacttgatagacgccaagaggcctgaattgttggaggcccttaataggtctgctatcattaaagacgacaaatag

(19) United States
(12) Patent Application Publication
(10) Pub. No.: US 2023/0220018 A1
(43) Pub. Date: Jul. 13, 2023

(54) HETEROLOGOUS DDP1 EXPRESSING PLANTS AND USES THEREOF

(71) Applicants: VIRGINIA POLYTECHNIC INSTITUTE AND STATE UNIVERSITY, Blacksburg, VA (US); VIRGINIA TECH INTELLECTUAL PROPERTIES, INC., Blacksburg, VA (US)

(72) Inventors: Glenda Gillaspy, Blacksburg, VA (US); Catherine Freed, Blacksburg, VA (US); Sarah Phoebe Williams, Blacksburg, VA (US); Zhiwu Wang, Blacksburg, VA (US)

(21) Appl. No.: 17/927,082

(22) PCT Filed: May 22, 2021
(86) PCT No.: PCT/US2021/033799

§ 371 Date: Nov. 22, 2022

Related U.S. Application Data
(60) Provisional application No. 63/028,970, filed on May 22, 2020.
(60) Provisional application No. 63/106,408, filed on Oct. 28, 2020.

(51) Int. Cl. C07K 14/415; C07K 14/39; C12N 15/82

(52) U.S. Cl. CPC C07K 14/415; C07K 14/39; C12N 15/8261

(57) ABSTRACT

Described herein are engineered cells and plants that contain a heterologous Diadenosine and Diphosphoinositol Polyphosphate Phosphohydrolase (DDP1) polypeptide, a heterologous DDP1 encoding polynucleotide, a vector or vector system comprising a heterologous DDP1 encoding polynucleotide, or a combination thereof. Also described herein are methods of making and using the engineered cells and plants described herein.

### CROSS-REFERENCE TO RELATED APPLICATIONS

This application claims the benefit of and priority to U.S. Provisional Pat. Application No. 63/028,970, filed on May 22, 2020, entitled “HETEROLOGOUS DDP1 EXPRESSING PLANTS AND USES THEREOF,” the contents of which is incorporated by reference herein in its entirety.

This application also claims the benefit of and priority to U.S. Provisional Pat. Application No. 63/106,408, filed on Oct. 28, 2020, entitled UTILIZING SLOW-RELEASING FERTILIZER PROCESSED FROM PHOSPHATE HYPERACCUMULATING PLANTS TO REMEDIATE PHOSPHATE POLLUTION,” the contents of which is incorporated by reference herein in its entirety.

### STATEMENT REGARDING FEDERALLY SPONSORED RESEARCH

This invention was made with government support under Grant No. MCB 1616038 awarded by National Science Foundation. The government has certain rights in the invention.

### SEQUENCE LISTING

This application contains a sequence listing filed in electronic form as an ASCII.txt file entitled VTIP-0260WP_ST25.txt, created on May 21, 2021 and having a size of 2,921 bytes (4 KB on disk). The content of the sequence listing is incorporated herein in its entirety.

### TECHNICAL FIELD

The subject matter disclosed herein is generally directed to engineered plants, and more specifically plants engineered for phytoremediation.

### BACKGROUND

Phosphate (Pi) is crucial for genetic maintenance, cellular function, and energy metabolism in plants as intra-and extracellular Pi pools regulate numerous plant signaling pathways (Kanno et al. Plant Cell Physiol 2016, 57, 690-706 and Kuo et al., The Plant Journal 2018, 95, 613-630). Pi is arguably the greatest plant growth-limiting macronutrient, making it the foundation for food production and security worldwide (Kuo et al., The Plant Journal 2018, 95, 613-630; Sattari et al., Proc Natl Acad Sci USA 2012, 109, 6348-6353; and Song et al., Front. Plant Sci. 2015, 6, doi:10.3389/fpls.2015.00796). While critically important, Pi is, unfortunately, scarce in most soils. There is a phosphate crises, with at most an estimated 300 year supply remaining in minable reserves. Ironically, there is simultaneously a phosphorous pollution is a widespread problem with where excessive application of fertilizer to agricultural land and urban areas leaches into aquatic environments. Thus, there exists a need for improved compositions, methods, and/or techniques for improved Pi utilization and management.

Citation or identification of any document in this application is not an admission that such a document is available as prior art to the present invention.

### SUMMARY

Described in certain example embodiments herein are engineered plant cells comprising a heterologous Diadenosine and Diphosphoinositol Polyphosphate Phosphohydrolase (DDP1) polypeptide, a heterologous DDP1 encoding polynucleotide, a vector or vector system comprising a heterologous DDP1 encoding polynucleotide, or a combination thereof.

In certain example embodiments herein, the plant cell expresses the heterologous DDP1 polypeptide, the heterologous DDP1 encoding polynucleotide, the vector or vector system comprising the heterologous DDP1 encoding polynucleotide, or a combination thereof.

In certain example embodiments herein, the heterologous DDP1 is a fungi DDP1 or a mammalian DDP1. In certain example embodiments herein, the fungi DDP1 is a yeast DDP1. In certain example embodiments herein, the yeast DDP1 is a DDP1 from the genus Saccharomyces, Candida, Zygosaccharomyces, Kluyveromyces, Babjeviella, Kazachstania, Torulaspora, Tetrapisispora, Lachancea, Naumovozyma, and related strains. In certain example embodiments herein, the DDP1 is a Saccharomyces cerevisiae DDP1.

In certain example embodiments herein, the DDP1 polypeptide is about 50-100% identical to SEQ ID NO: 1.

In certain example embodiments herein, the DDP1 encoding polynucleotide is about 50-100% identical to SEQ ID NO: 2.

Described in certain example embodiments herein are engineered plants comprising: one or more cells as in any one of the preceding paragraphs and/or as described in greater detail elsewhere herein.

In certain example embodiments herein, the engineered plant is an engineered monocotyledonous plant or an engineered dicotyledonous plant.

In certain example embodiments herein, the engineered dicotyledonous plant belongs to the order Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violates, Salicales, Capparales, Ericales, Diapensales, Ebenales, Brassicales, Primulales, Rosales, Fabales, Podostemales, Haloragales, Myrtales, Comales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, or Asterales.

In certain example embodiments herein, the engineered monocotyledonous plant belongs to the order Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, Orchid ale, Pinales, Ginkogoales, Cycadales, Araucariales, Cupressales or Gnetales.

In certain example embodiments herein, the engineered plant is a species of Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Thlaspi, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, or Pseudotsuga.

In certain example embodiments herein, the plant is a grain crop plant (e.g., wheat, maize, rice, millet, barley), a fruit crop plant (e.g., tomato, apple, pear, strawberry, orange), forage crops (e.g., alfalfa), a root vegetable crop plant (e.g., carrot, potato, sugar beets, yam), a leafy vegetable crop plant (e.g., lettuce, spinach); a flowering crop plant (e.g., petunia, rose, chrysanthemum), a conifers or pine tree (e.g., pine fir, spruce); a plant used in phytoremediation (e.g., heavy metal accumulating plants); an oil crop plant (e.g., sunflower, rape seed), a ground cover, a turf or other grass, or a plant typically used for experimental purposes (e.g., Arabidopsis).

In certain example embodiments herein, the engineered plant is an angiosperm or a gymnosperm plant.

In certain example embodiments herein, the engineered plant is acacia, alfalfa, amaranth, apple, apricot, artichoke, ash tree, asparagus, avocado, banana, barley, beans, beet, birch, beech, blackberry, blueberry, broccoli, Brussel’s sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pennycress, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, or zucchini.

In certain example embodiments herein, the engineered plant is a turfgrass.

In certain example embodiments herein, the engineered plant is an algae.

In certain example embodiments herein, the engineered plant is an algae from the phyla Rhodophyta (red algae), Chlorophyta (green algae), Phaeophyta (brown algae), Bacillariophyta (diatoms), Eustigmatophyta, a dinoflagellates, or the prokaryotic phylum Cyanobacteria (blue-green algae).

In certain example embodiments herein, the engineered algae the species of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, or Trichodesmium.

In certain example embodiments herein, the engineered plant is a fern, moss, or liverwort.

In certain example embodiments herein, the engineered cell of any one of the preceding paragraphs and/or as described elsewhere herein or the engineered plant of any of the preceding paragraphs and/or as described elsewhere herein the engineered cell, the engineered plant, or both comprise a DDP1 encoding polynucleotide stably integrated into the genome of the engineered cell.

In certain example embodiments herein, the engineered cell of any one of the preceding paragraphs and/or as described elsewhere herein or the engineered plant of any of the preceding paragraphs and/or as described elsewhere herein the engineered cell, the engineered plant, or both comprise a DDP1 encoding polynucleotide that is transiently expressed in the engineered cell, engineered plant, or both.

In certain example embodiments herein, the engineered plant or cell thereof of any of the preceding paragraphs and/or as described elsewhere herein has one or more modulated observable traits as compared to an unmodified plant.

In certain example embodiments herein, the engineered plant or cell thereof of any of the preceding paragraphs and/or as described elsewhere herein the engineered plant has increased growth and/or or performance in at least one economically important trait, optionally wherein the trait comprises improved Pi utilization, accumulation, and/or storage, growth, fruit yield, flower yield, hardiness, stress tolerance, or any combination thereof.

Described in certain example embodiments herein are methods comprising modifying a plant cell such that the plant cell comprises a heterologous Diadenosine and Diphosphoinositol Polyphosphate Phosphohydrolase (DDP1) polypeptide, a heterologous DDP1 encoding polynucleotide, a vector or vector system comprising a heterologous DDP1 encoding polynucleotide, or any combination thereof.

In certain example embodiments herein, modifying comprises delivering a polynucleotide having a sequence that is about 50-100% identical to SEQ ID NO: 2 to the cell, delivering a polypeptide having a sequence that is about 50-100% identical to SEQ ID NO: 1 to the cell, or both.

Described in certain example embodiments herein are methods comprising growing, propagating, harvesting, and/or cultivating a plant as in any one of the preceding paragraphs and/or as described elsewhere herein.

Described in certain example embodiments herein are methods of removing Pi from a soil or water, comprising growing, propagating, harvesting, and/or cultivating a plant as in any one of the preceding paragraphs and/or as described elsewhere herein in the soil or water.

Described in certain example embodiments herein are methods of producing biochar, the method comprising carbonizing biomass from a plant as in any one of the preceding paragraphs and/or as described elsewhere herein by a suitable process to form the biochar.

Described in certain example embodiments herein are biochars and/or fertilizers produced from a DDP1 overexpressing plant.

In certain example embodiments herein, the DDP1 overexpressing plant is as in any one of the preceding paragraphs and/or as described elsewhere herein.

In certain example embodiments herein, the biochar releases phosphorus at a rate 1-100 times slower as compared to biochar made from a wild-type of non-DDP1 expressing control plant.

Described in certain example embodiments herein are methods of applying nutrients and/or a fertilizer to a soil, the method comprising applying an amount of biomass from a DDP1 overexpressing plant and/or a biochar produced from a DDP1 overexpressing plant to the soil. In certain example embodiments, the biomass is fresh biomass. In certain example embodiments herein, the DDP1 over expressing plant is as in any one of the preceding paragraphs and/or as described elsewhere herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1C - Battery of severe physiological phenotypes in severe DDP1 OX transgenics. (FIG. 1A) T₄ DDP1 OX transgenic with matched control. (FIG. 1B) A closer view of a severe DDP1 OX bolt with flower abortions at the base and small siliques at bolt top. (FIG. 1C) Rosettes from 5 week-old severe DDP1 OX transgenics grown on soil. Yellowing lesions at leaf tips are characteristic of all severe DDP1 OX transgenics.

FIGS. 2A-2D - (FIG. 2A) Rosette diameters of selected DDP1 OX severe and nonsevere lines after about 2.5 weeks of growth. DDP1-H is considered non-severe as it is more WT-like. DDP1-I and DDP1-A are considered severe, exhibiting curled leaves and lesions of the tips of the leaves. Scale bar = 10 mm. (FIG. 2B) Western blot showing DDP1 accumulation in the selected lines. DDP1-I has the highest accumulation, then DDP1-A, and lastly, DDP1-H. No accumulation in WT. The PonceauS shows RUBISCO in all four samples (FIG. 2C). (FIG. 2D) Rosette diameters of representative transgenics of all DDP1 overexpressing lines and mutants for up to 7 weeks of growth. Scale bar = 1 cm. FIG. 2D can be compared with FIG. 20.

FIGS. 3A-3B - (FIG. 3A) WT, DDP1-I, and ipk1- under varying Pi levels after 43 days of growth. (FIG. 3B) Reproductive growth of DDP1-I under deplete and replete Pi after 50 days.

FIGS. 4A-4D - Inositol Phosphate Profiles of WT and DDP1 OX Transgenics. (FIG. 4A) 2-week old seedlings were grown on semi-solid 0.5X MS media with 0.2% agar and transferred them to media containing 100 µCi [³H]-myo-inositol for 4 days (n=2). InsPs were extracted, separated on an anion exchange HPLC machine, and quantified using a scintillation counter. (FIG. 4B) The ratio of InsP₆ and PP-InsPs in DDP1 OX lines to WT (n=5). (FIGS. 4C-4D) Ratio of InsP₆ to InsP₇ (FIG. 4C) and ratio of InsP₇ to InsP₈ (FIG. 4D) in WT and DDP1 OX (n=2-5).

FIGS. 5A-5L - (FIGS. 5A-5C) Stable DDP1-GFP expression in Arabidopsis (DDP1-I) leaves (FIGS. 5A-B) and roots (FIG. 5C) after 3 weeks of growth using confocal microscopy. Scale bar = 50 µm. (FIG. 5D) DDP1-GFP transient expression in N. benthamiana leaves 48 hours post-infiltration. Green represents DDP1-GFP and red represents autofluorescence. (FIG. 5E) An unifiltrated N. benthamiana leaf. (FIG. 5F) Transient expression of YFP-DDP1 in N. benthamiana leaves 48 hours post-infiltration. (FIGS. 5G-5L) N. benthamiana leaves co-infiltrated with DDP1-GFP and mCherry (G-1) or DDP1-GFP and ER-mCherry (FIGS. 5J-5L). Images in FIGS. 5A-5B and FIGS. 5D-5L are presented as maximum intensity projections from confocal Z-stack optical sections.

FIGS. 6A-6B - (FIG. 6A) Shoot Pi content of 28-day old transgenics and mutants grown on soil, n=3. Analyzed using a one-way ANOVA and Tukey-Kramer HSD. Connecting letters report denotes significant differences between genotypes; genotypes with the same letter are not significant from each other. (FIG. 6B) qPCR analysis of PSR genes. Gene expression was measured from RNA in 2-week old seedlings, n=1

FIGS. 7A-7B - Gradient phenotypes of selected independent DDP1 OX T₁ transgenic lines from the second screen of 40 isolated lines. This gradient is representative of two independent Arabidopsis screens. (FIG. 7A) Plant size and physiology of 8 independent DDP1 OX lines from the second screen. (FIG. 7B) Closer view of rosette diameters of 10 independent lines from the screen.

FIGS. 8A-8B - Gradient phenotype assessment of DDP1 OX transgenics based on DDP1-GFP accumulation, plant size, and seeds collected. DDP1-GFP accumulation is assessed through western blotting. The PonceauS shows RuBisCo accumulation in all plants as a positive control. The seeds in the Eppendorf tubes crudely represent the number of seeds collected from each individual plant. (FIG. 8A) Evaluates intermediately severe phenotypes of four independent T₂ DDP1 OX transgenics. (FIG. 8B) Evaluates DDP1-GFP accumulation in a segregating T₂ siblings from one independent DDP1 OX line.

FIG. 9 - Additional inositol phosphate profile of WT and DDP1 OX transgenics. Performed under the same conditions as FIGS. 4A-4D.

FIG. 10 - Alternative view of WT and DDP1-I inositol phosphate profiles from FIGS. 4A-4D and 8A-8B. The graphs show alterations in InsP₆ between the two replicates.

FIG. 11 - Inositol phosphate profiles of WT and DDP1-I. One hundred 2-week old seedlings were grown on semi-solid 0.5X-MS media with 0.2% agar and transferred them to media containing 200 µCi [3H] myo-inositol for 4 days (n=2).

FIGS. 12A-12L - DDP1-GFP localization in Arabidopsis epidermal cells. Mature leaves 3-week old soil grown Arabidopsis were imaged. WT (FIGS. 12A-12C), DDP 1-H (FIGS. 12D-12F), DDP1-I (FIGS. 12G-12I), and DDP1-A (FIGS. 12J-12L).

FIGS. 13A-13F - Time course of DDP1-GFP expression. DDP1-GFP was transiently expressed in N. benthamiana leaves and imaged 24 (FIGS. 13A-13C) and 48 (FIGS. 13D-13F) hours post-infiltration using confocal microscopy. Scale bar = 50 µm.

FIGS. 14A-14I - Transient DDP1-GFP expression in N. benthamiana leaves 48 hours post-infiltration using confocal microscopy. DDP1-GFP (FIGS. 14A, 14D, 14G) was co-expressed with free mCherry (FIGS. 14B, 14E) and ER-mCherry (FIG. 14H). FIG. 14C and FIG. 14F show the DDP1-GFP + free mCherry merge and (FIG. 14I) is the DDP1-GFP + ER-mCherry merge. Scale bar = 50 µm.

FIGS. 15A-15I - N. benthamiana leaves co-infiltrated with YFP-DDP1 (FIGS. 15A, 15D, 15G) and mCherry (FIGS. 15B, 15E, 15H). Cells were imaged at 24 (FIGS. 15A-15C), 48 (FIGS. 15D-15F), and 72 (FIGS. 15G-15H) hours post-infiltration using confocal microscopy. Scale bar = 50 µm.

FIGS. 16A-16F - Phenotypes of Pennycress DDP1 overexpressors (PcDDP1 OX). (FIG. 16A) Hygromycin-resistant PcDDP1OX seedling growing on selective media after 3 weeks of growth. (FIG. 16B) Accumulation of leaf lesions in PcDDP1OX transplant. (FIG. 16C) Mature PcDDPIOX transgenic, DDP1-B, physiology after 6 weeks of growth, transplanted on soil. (FIG. 16D) Stable overexpression of YFP-DDP1 in PcDDP1 OX leaf epidermal cells. Expressional patterns exhibitYFP-DDP1 localizes to the guard cell and epidermal cell nuclei and cytoplasm. (FIG. 16E) Semi-quantitative PCR showing DDP1-B has been transformed with YFP-DDP1, at roughly 1 kB. Ladder (M), non-template control (-), WT pennycress (WT), DDP1-B transformant (Trans), and DDP1 plasmid positive control (+). (FIG. 16F) Total Pi accumulation in leaf tissue from Arabidopsis and pennycress WT and DDP1 OX transgenics. There is an 8-10 fold increase in both Pennycress and Arabidopsis DDP1 OX transgenics (n=1-3).

FIG. 17 - Average total Phosphorus content in biochar from WT and AtDDP1 OX. Phosphorus content analyzed using Inductively coupled plasma mass spectrometry on biochar from 5-week old Arabidopsis plants. Total tissue divided into leaves and stems/shoots/siliques (reproductive shoots). Mean with SD, n=1-2.

FIG. 18 - Percentage of Pi released from biochar in water after 24 hours in WT and AtDDP1 OX. Mean with SD, n = 1-2.

FIGS. 19A-19D - Inositol Phosphate Profiles in Arabidopsis WT and DDP1 OX Transgenics. (FIG. 19A) 2-week old seedlings were grown on semi-solid 0.5 X MS media with 0.2% agar and transferred to media containing 100 µCi of [³H]-myo-inositol for 4 days (n=2-3 independent biological replicates). InsPs were extracted, separated on an anion exchange HPLC machine, and quantified using a scintillation counter. These traces are representative of 2-3 independent replicates per genotype. (FIG. 19B) InsP6/InsP7, (FIG. 19C) InsP7/InsP8, and (FIG. 19D) InsP7/InsP8 ratios from InsP profiles; data analyzed using students T-Test; “*” used for WT versus transgenic; * denotes p < 0.05; ** denotes p< 0.005, n=2-3.

FIG. 20 - Shoot Pi accumulation over time in Arabidopsis plants from 2-7 weeks of growth on soil. n=3 per genotype per timepoint. * denotes p<0.05, ** denotes p<0.005, analyzed using a one-way ANOVA and Tukey-Kramer HSD for each individual time point.

FIGS. 21A-21B - Arabidopsis biochar qualities. (FIG. 21A) Total P content and (FIG. 21B) percent Pi (from total P content) released in water after 1, 3, 6, and 16-day incubation periods in biochar samples produced from 6-week-old shoot tissue, n= 2 technical replicates, error bars = SD.

FIGS. 22A-22B - DDP1 overexpressing phenotype. (FIG. 22A) Aborting siliques in 5-week-old DDP1 OX (DDP1-I). White arrowheads indicate aborting siliques, yellow arrowheads mark yellowing cauline leaves. Scale bar = 1 cm. (FIG. 22B) Leaf lesions in DDP1 OX (DDP1-I) 4-weeks-old; scale bar = 1 cm.

FIGS. 23A-23B - Inorganic phosphate accumulation in and release from processed plants from two species engineered to express a heterologous DDP1.(FIG. 23A) Inorganic phosphate content of two different transgenic plant species, grown on soil, n=1-3. There is an 8-10 fold increase in both transgenics, regardless of species type. Asterisks denote values are significantly different. (FIG. 23B) Percentage of inorganic phosphate released from processed plants in water after 24 hours in WT and the transgenic line. Lower numbers indicate slow-release of phosphate. Mean with SD, n = 1-2.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y″’, where ‘x’ and ‘y’ are numerical values, includes “about ‘x’ to about ‘y”’.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F.M. Ausubel et al. eds.); the series Methods in Enzymology (Academic Press, Inc.): PCR 2: A Practical Approach (1995) (M.J. MacPherson, B.D. Hames, and G.R. Taylor eds.): Antibodies, A Laboratory Manual (1988) (Harlow and Lane, eds.): Antibodies A Laboraotry Manual, 2^{nd} edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/-10% or less, +/-5% or less, +/-1 % or less, and +/-0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. Biological samples include cell cultures, tissues, fruits or other products produced from a biological entity, seeds, pods, tubers, rhizomes, and other plant components and/or parts plant samples Biological samples may be obtained from a plant for example by puncture, cutting, pressing, or other collecting or sampling procedures.

The terms “subject,” “individual,” are used interchangeably herein to refer to a single individual unit of a population, such as of plants and/or animals or a cell thereof. Tissues, cells and their progeny of a biological entity obtained in vivo or cultured in vitro are also encompassed.

As used herein, “control” can refer to an alternative subject or sample used in an experiment for comparison purpose and included to minimize or distinguish the effect of variables other than an independent variable.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “culturing” can refer to maintaining cells under conditions in which they can proliferate and avoid senescence as a group of cells. “Culturing” can also include conditions in which the cells also or alternatively differentiate.

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “modulate” broadly denotes a qualitative and/or quantitative alteration, change or variation in that which is being modulated. Where modulation can be assessed quantitatively — for example, where modulation comprises or consists of a change in a quantifiable variable such as a quantifiable property of a cell or where a quantifiable variable provides a suitable surrogate for the modulation — modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in aspects modulation may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In aspects, modulation may encompass a decrease or reduction in the value of the measured variable by about 5 to about 100%. In some aspects, the decrease can be about 5%,10%, 20%, 30%, 40%, 50%, 60%, 70%, 80%, 90%, 95%, 96%, 97%, 98%, 99% to about 100%, compared to a reference situation or suitable control without said modulation. In aspects, modulation may be specific or selective, hence, one or more desired phenotypic aspects of a cell or cell population may be modulated without substantially altering other (unintended, undesired) phenotypic aspect(s).

As used herein, “nucleic acid,” “nucleotide sequence,” and “polynucleotide” can be used interchangeably herein and can generally refer to a string of at least two base-sugar-phosphate combinations and refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “observable trait” refers to any characteristic of a cell, population of cell, tissue, organ, organ system, and/or organism that is measurable or otherwise observable. An observable trait can be, for example, gene expression, protein expression, epigenetic status or signature, functionality, morphology, temporal and/or spatial localization. An observable trait or traits can define a phenotype.

As used interchangeably herein, “operatively linked” and “operably linked” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “overexpressed” or “overexpression” refers to an increased expression level of an RNA and/or protein product encoded by a gene as compared to the level of expression of the RNA or protein product in a normal or control cell. The amount of increased expression as compared to a normal or control cell can be about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.2, 2.4, 2.6, 2.8, 3.0, 3.3, 3.6, 3.9, 4.0, 4.4, 4.8, 5.0, 5.5, 6, 6.5, 7, 7.5, 8.0, 8.5, 9, 9.5, 10, 11, 12, 13, 14, 15, 16, 17, 18, 19, 20, 25, 30, 35, 40, 45, 50, 60, 70, 0, 90, 100 fold or more greater than the normal, unmodified, or control cell. “increased expression” or “overexpression” are both used to refer to an increased expression of a gene, such as a transgene or gene product thereof in a sample as compared to the expression of said gene or gene product in a suitable control. The term “ increased expression” refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control in some embodiments herein.

As used herein, “plasmid” refers to a non-chromosomal double-stranded DNA sequence including an intact “replicon” such that the plasmid is replicated in a host cell.

As used herein, “positive control” refers to a “control” that is designed to produce the desired result, provided that all reagents are functioning properly and that the experiment is properly conducted.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body’s cells, tissues, and organs.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein “reduced expression” or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “substantial” and “substantially,” specify an amount of between 95% and 100%, inclusive, between 96% and 100%, inclusive, between 97% and 100%, inclusive, between 98% 100%, inclusive, or between 99% 100%, inclusive.

As used herein, the term “vector” or is used in reference to a vehicle used to introduce an exogenous nucleic acid sequence into a cell. A vector may include a DNA molecule, linear or circular (e.g. plasmids), which includes a segment encoding an RNA and/or polypeptide of interest operatively linked to additional segments that provide for its transcription and optional translation upon introduction into a host cell or host cell organelles. Such additional segments can include promoter and/or terminator sequences, and can also include one or more origins of replication, one or more selectable markers, an enhancer, a polyadenylation signal, etc. Expression vectors are generally derived from yeast or bacterial genomic or plasmid DNA, or viral DNA, or may contain elements of both. Expression vectors can be adapted for expression in prokaryotic or eukaryotic cells. Expression vectors can be adapted for expression in mammalian, fungal, yeast, or plant cells. Expression vectors can be adapted for expression in a specific cell type via the specific regulator or other additional segments that can provide for replication and expression of the vector within a particular cell type.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

As used herein, the terms “weight percent,” “wt%,” and “wt. %,” which can be used interchangeably, indicate the percent by weight of a given component based on the total weight of a composition of which it is a component, unless otherwise specified. That is, unless otherwise specified, all wt% values are based on the total weight of the composition. It should be understood that the sum of wt% values for all components in a disclosed composition or formulation are equal to 100. Alternatively, if the wt% value is based on the total weight of a subset of components in a composition, it should be understood that the sum of wt% values the specified components in the disclosed composition or formulation are equal to 100.

All publications, published patent documents, and patent applications cited herein are hereby incorporated by reference to the same extent as though each individual publication, published patent document, or patent application was specifically and individually indicated as being incorporated by reference.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

### OVERVIEW

Phosphate (Pi) is crucial for genetic maintenance, cellular function, and energy metabolism in plants as intra-and extracellular Pi pools regulate numerous plant signaling pathways [1,2]. Pi is arguably the greatest plant growth-limiting macronutrient, making it the foundation for food production and security worldwide [2-4]. While critically important, Pi is, unfortunately, scarce in most soils. Under deplete Pi conditions, plants employ molecular mechanisms, known as Pi starvation responses (PSR), to reprioritize growth patterns to increase environmental uptake, and redistribute Pi from existing cells [5,6]. The PSR is modulated by complex signaling networks and while its regulation is not completely understood, emerging evidence suggests inositol pyrophosphates (PP-InsPs) are the key regulators of the PSR.

PP-InsPs and their precursors, inositol phosphates (InsPs), are crucial for plant development, energy metabolism, and stress responses[7,8]. InsPs consist of a myo-inositol ring with hydroxyl groups that are sequentially phosphorylated. The number and position of phosphate moieties on the ring allow the plant cells to convey different messages. InsP₆, also known as phytate when chelated with metals, is the most abundant InsP species found in plants and is important for Pi storage as well as signaling [9-11]. InsP₆ was found to regulate plant auxin signaling through binding interacts with transport inhibitor 1 (TIR1) [12]. InsP₆ can be further phosphorylated form InsP₇, which is hypothesized to bind to the jasmonate receptor, as well as InsP₈ [13]. However, the roles of these molecules in plant signaling is unclear. PP-InsPs have recently been implicated in Pi sensing [14-17]. Thus, there exists a need for improved understanding, compositions, methods, and/or techniques for improved Pi utilization and management.

With that said, embodiments disclosed herein can provide engineered plants and cells thereof comprising a heterologous Diadenosine and Diphosphoinositol Polyphosphate Phosphohydrolase (DDP1). The engineered plants and cells thereof described herein can have an altered phenotype as to one or more characteristics, such as those associated with Pi storage, utilization, growth, fruit yield, flower yield, hardiness, stress tolerance, and combinations thereof. Also described herein are methods of generating and using the engineered plants expression a heterologous DDP1. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### ENGINEERED DDP1 EXPRESSING PLANT CELLS AND PLANTS

Described herein are engineered plant cells and plants including and/or expressing a heterologous Diadenosine and Diphosphoinositol Polyphosphate Phosphohydrolase (DDP1) polypeptide, a heterologous DDP1 encoding polynucleotide, a vector or vector system containing a heterologous DDP1 encoding polynucleotide, or a combination thereof.

### DDP1 Polypeptides, Polynucleotides, and Vectors

In some embodiments, plants can contain and/or express in one, one or more, or all of its cells one or more heterogonous DDP1 polypeptides, polynucleotides, vectors, vector systems, or any combination thereof. In some embodiments, one or more plant cells contains and/overexpress the heterologous DDP1 polypeptide, the heterologous DDP1 encoding polynucleotide, the vector or vector system containing the heterologous DDP1 encoding polynucleotide, or a combination thereof.

DDP1 polypeptides, polynucleotides and vectors and/or vectors systems containing the DDP1 polynucleotides and/or capable of expressing one or more of the DDP1 polynucleotides are described herein. Any DDP1 not native to the host (or background) plant is suitable for use in the context of the DDP-expressing plants described herein.

In some embodiments, the heterologous DDP1 can be a homologue or orthologue of S. cerevisiae DDP1 (e.g. SEQ ID No: 1 or 2). The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 Apr;22(4):359-66. doi: 10.1002/pro.2225.). Homologous proteins may but need not be structurally related, or are only partially structurally related.

Sequence homologies may be generated by any of a number of computer programs known in the art, for example BLAST or FASTA, etc. A suitable computer program for carrying out such an alignment is the GCG Wisconsin Bestfit package (University of Wisconsin, U.S.A; Devereux et al., 1984, Nucleic Acids Research 12:387). Examples of other software than may perform sequence comparisons include, but are not limited to, the BLAST package (see Ausubel et al., 1999 ibid - Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, it is preferred to use the GCG Bestfit program. Percentage (%) sequence homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity. However, these more complex methods assign “gap penalties” to each gap that occurs in the alignment so that, for the same number of identical amino acids, a sequence alignment with as few gaps as possible — reflecting higher relatedness between the two compared sequences — may achieve a higher score than one with many gaps. “Affinity gap costs” are typically used that charge a relatively high cost for the existence of a gap and a smaller penalty for each subsequent residue in the gap. This is the most commonly used gap scoring system. High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is -12 for a gap and -4 for each extension. Calculation of maximum % homology therefore first requires the production of an optimal alignment, taking into consideration gap penalties. A suitable computer program for carrying out such an alignment is the GCG Wisconsin Bestfit package (Devereux et al., 1984 Nuc. Acids Research 12 p387). Examples of other software than may perform sequence comparisons include, but are not limited to, the BLAST package (see Ausubel et al., 1999 Short Protocols in Molecular Biology, 4^{th} Ed. - Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health). Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix - the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62. Alternatively, percentage homologies may be calculated using the multiple alignment feature in DNASIS™ (Hitachi Software), based on an algorithm, analogous to CLUSTAL (Higgins DG & Sharp PM (1988), Gene 73(1), 237-244). Once the software has produced an optimal alignment, it is possible to calculate % homology, preferably % sequence identity. The software typically does this as part of the sequence comparison and generates a numerical result. The sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. However, it is more useful to include mutation data as well. The sets of amino acids thus derived are likely to be conserved for structural reasons. These sets may be described in the form of a Venn diagram (Livingstone C.D. and Barton G.J. (1993) “Protein sequence alignments: a strategy for the hierarchical analysis of residue conservation” Comput. Appl. Biosci. 9: 745-756) (Taylor W.R. (1986) “The classification of amino acid conservation” J. Theor. Biol. 119; 205-218). Conservative substitutions may be made, for example according to the table below which describes a generally accepted Venn diagram grouping of amino acids.

In some embodiments, the heterologous DDP1 is a fungi DDP1 or a mammalian DDP1. In some embodiments, the fungi DDP1 is a yeast DDP1. Ins some embodiments, the yeast DDP1 is a DDP1 from the genus Saccharomyces, Candida, Zygosaccharomyces, Kluyveromyces, Babjeviella, Kazachstania, Torulaspora, Tetrapisispora, Lachancea, Naumovozyma and related yeast strains. In some embodiments, the related yeast strain’s DDP1 or other protein has about 60, 65, 70, 76, 80, 85, 90, 95, 96, 97, 98, 99 or about 99.9 identity with S. cerevisiae DDP1 (e.g., SEQ ID NO. 1). In some embodiments, the heterologous DDP1 is a Saccharomyces cerevisiae DDP1.

In some embodiments, the heterologous DDP1 polypeptide is about 50-100% identical to SEQ ID NO: 1. In some embodiments, the heterologous DDP1 polypeptide is about 50, to/or 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 % identical to SEQ ID NO: 1.

The DDP1 polypeptide can be encoded by a heterologous DDP1 encoding polynucleotide. In some embodiments the sequence is codon optimized for expression in a plant cell. Methods of codon optimization include those described in Kwon KC, et al., Codon Optimization to Enhance Expression Yields Insights into Chloroplast Translation, Plant Physiol. 2016 Sep;172(1):62-77.

In some embodiments, the heterologous DDP1 encoding polynucleotide is about 50-100% identical to SEQ ID NO: 2. In some embodiments, the heterologous DDP1 encoding polynucleotide is about 50, to/or 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 % identical to SEQ ID NO: 2.

### Vectors and Vector Systems

Also provided herein are vectors that can contain one or more of the heterologous DDP1 encoding polynucleotides described herein. The vectors can be useful in producing, for example, bacterial, plant cells, and/or transgenic plants that can contain, replicate, and/or express a heterologous DDP1 described herein. Within the scope of this disclosure are vectors containing one or more of the polynucleotide sequences described herein. One or more of the polynucleotides that are part of the heterologous DDP1 encoding polynucleotides described herein can be included in a vector or vector system. The vectors and/or vector systems can be used, for example, to express one or more of the polynucleotides in a cell, such as a plant cell or agrobacterium or to produce particles such as viral particles that can be used to generate transgenic cells and/or plants. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g. a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other aspects of the vectors and vector systems are described elsewhere herein.

In some embodiments, the vector can be a bicistronic vector. In some aspects, a bicistronic vector can be used for expression one or more DPP1 or other (e.g., reporter) polynucleotides and/or one or more regions or domains thereof described herein.

### Cell-based Vector Amplification and Expression

Vectors can be designed for amplification, propagation, and expression of one or more elements of the heterologous DDP1 encoding polynucleotides described herein (e.g. nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, mammalian cells, and more particularly plant cells. The vectors can be viral-based or non-viral based. In some embodiments, the suitable host cell is a eukaryotic cell. In some embodiments, the suitable host cell is a suitable bacterial cell. Suitable bacterial cells include, but are not limited to bacterial cells from the bacteria of the species Escherichia coli. Many suitable strains of E. coli are known in the art for expression of vectors. These include, but are not limited to Pirl, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some aspects, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R.G. and Gleeson, M.A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2µ plasmids, yeast integrative plasmids, yeast replicative plasmids, shuttle vectors, and episomal plasmids.

In some aspects, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some aspects, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Vectors may be introduced and propagated in a prokaryote or prokaryotic cell. In some aspects, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some aspects, a prokaryote is used to amplify copies of a vector and express one or more nucleic acids, such as to provide a source of one or more proteins for delivery to a host cell or host organism.

In some aspects, the vector can be a fusion vector or fusion expression vector. In some aspects, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some aspects, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some aspects, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, where one or more DDP1 polynucleotides, functional domains thereof, and/or one or more additional polynucleotides are included and/or expressed (e.g. a reporter polynucleotide), each be operably linked to separate regulatory elements on the same or separate vectors. In some aspects, two or more of the elements expressed from the same or different regulatory element(s), can be combined in a single vector, with one or more additional vectors providing any components of the system not included in the first vector. Encoding polynucleotides (DDP1 or others) that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more DDP1 proteins and/or functionals domains thereof and/or one or more other genes (e.g. reporter gene), embedded within one or more intron sequences (e.g., each in a different intron, two or more in at least one intron, or all in a single intron). In some embodiments, the DDP1 proteins and/or functionals domains thereof and/or one or more other genes (e.g. reporter gene) can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g. molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

Where a vector or vector system is provided that contains the heterologous DDP1 encoding polynucleotide, the encoding polynucleotide can be operatively coupled to one or more regulatory elements. In some embodiments, the regulatory element can drive ubiquitous expression. In some embodiments, the regulatory element can drive or control cell or tissue specific expression. In some embodiments, the regulatory element can drive conditional or inducible expression. In some embodiments, the heterologous DDP1 encoding polynucleotide is operatively coupled to a plant promoter.

In aspects, the polynucleotides and/or vectors thereof described herein (such as the DDP1 encoding polynucleotides) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5’ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some aspects, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some aspects, the vector can contain a minimal promoter. In some aspects, the minimal promoter is the Mecp2 promoter, tRNA promoter, or U6. In a further embodiment, the minimal promoter is tissue specific. In some aspects, the length of the vector polynucleotide the minimal promoters and polynucleotide sequences is less than 4.4 Kb.

To express a polynucleotide, the vector can include one or more transcriptional and/or translational initiation regulatory sequences, e.g. promoters, that direct the transcription of the gene and/or translation of the encoded protein in a cell. In some aspects a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some aspects, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some aspects, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer1l4), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment).The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the heterologous DDP1 encoding polynucleotides described herein are typically placed under control of a plant promoter, i.e. a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the heterologous DDP1 encoding polynucleotides are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in expression of the heterologous DDP1 encoding polynucleotides are found in Kawamata et al., (1997) Plant Cell Physiol 38:792-803; Yamamoto et al., (1997) Plant J 12:255-65; Hire et al, (1992) Plant Mol Biol 20:207-18,Kuster et al, (1995) Plant Mol Biol 29:759-72, and Capana et al., (1994) Plant Mol Biol 25:681 -91.

Further exemplary plant promoters suitable to drive expression of the heterologous DDP1 encoding polynucleotides include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18,Kuster et al, (1995) Plant Mol Biol 29:759-72, and Capana et al., (1994) Plant Mol Biol 25:681 -91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc), or light inducible systems (Phytochrome, LOV domains, or cryptochrome)., such as a Light Inducible Transcriptional Effector (LITE) that direct changes in transcriptional activity in a sequence-specific manner. The components of a light inducible system may include one or more heterologous DDP1 encoding polynucleotides described herein, a light-responsive cytochrome heterodimer (e.g. from Arabidopsis thaliana), and a transcriptional activation/repression domain. In some aspects, the vector can include one or more of the inducible DNA binding proteins provided in PCT publication WO 2014/018423 and U.S. Publications, 2015/0291966, 2017/0166903, 2019/0203212, which describe e.g. aspects of inducible DNA binding proteins and methods of use and can be adapted for use with the present invention.

In some aspects, transient or inducible expression can be achieved by including, for example, chemical-regulated promotors, i.e. whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

Where transient expression of an encoding polynucleotide described herein is desired, transient expression may be achieved using suitable vectors. Exemplary vectors that may be used for transient expression include a pEAQ vector (may be tailored for Agrobacterium-mediated transient expression) and Cabbage Leaf Curl virus (CaLCuV), and vectors described in Sainsbury F. et al., Plant Biotechnol J. 2009 Sep;7(7):682-93; and Yin K et al., Scientific Reports volume 5, Article number: 14926 (2015).

A plant promoter is capable of initiating transcription in plant cells, whether or not its origin is a plant cell. The use of different types of promoters is envisaged.

In some aspects, the vector or system thereof can include one or more elements capable of translocating and/or expressing an heterologous DDP1 encoding polynucleotide to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the heterologous DDP1 encoding polynucleotides can be operably linked, fused to, or otherwise modified to include a polynucleotide that encodes or is a selectable marker or tag, which can be a polynucleotide or polypeptide. In some aspects, the polynucleotide encoding a polypeptide selectable marker can be incorporated with the heterologous DDP1 encoding polynucleotides polynucleotide such that the selectable marker polypeptide, when translated, is inserted between two amino acids between the N- and C-terminus of the heterologous DDP1 polypeptide or at the N- and/or C-terminus of the heterologous DDP1. In some aspects, the selectable marker or tag is a polynucleotide barcode or unique molecular identifier (UMI).

It will be appreciated that the polynucleotide encoding such selectable markers or tags can be incorporated into a heterologous DDP1 encoding polynucleotide described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG-and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more heterologous DDP1 polypeptides described herein via suitable linker, such as a glycine or glycine serine linkers which are known in the art.

The vector or vector system can include one or more polynucleotides encoding one or more targeting moieties. In some aspects, the targeting moiety encoding polynucleotides can be included in the vector or vector system, such as a viral vector system, such that they are expressed within and/or on the virus particle(s) produced such that the virus particles can be targeted to specific cells, tissues, organelles, etc. In some aspects, the targeting moiety encoding polynucleotides can be included in the vector or vector system such that the heterologous DDP1 polynucleotides and/or products expressed therefrom include the targeting moiety and can be targeted to specific cells, tissues, organelles, etc. In some aspects, such as non-viral carriers, the targeting moiety can be attached to the carrier (e.g. polymer, lipid, inorganic molecule etc.) and can be capable of targeting the carrier and any attached or associated heterologous DDP1 s polynucleotide(s) to specific cells, tissues, organelles, etc.

### Cell-Free Vector and Polynucleotide Expression

In some aspects, the polynucleotide encoding a DDP1 can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g. translation of a purified polyribonucleotide) or linked/coupled to transcription. In some aspects, the cell-free (or in vitro) translation system can include extracts from rabbit reticulocytes, wheat germ, and/or E. coli. The extracts can include various macromolecular components that are needed for translation of exogenous RNA (e.g. 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g. reticulocyte lysates and wheat germ extracts). Some translation systems can utilize a DNA template as a starting material (e.g. E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the heterologous DDP1 encoding polynucleotides described herein can be codon optimized. In some aspects, one or more polynucleotides contained in a vector (“vector polynucleotides”) described herein that are in addition to an optionally codon optimized the heterologous DDP1 encoding polynucleotides described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar 25;257(6):3026-31. As to codon usage in plants including algae, reference is made to Codon usage in higher plants, green algae, and cyanobacteria, Campbell and Gowri, Plant Physiol. 1990 Jan; 92(1): 1-11.; as well as Codon usage in plant genes, Murray et al, Nucleic Acids Res. 1989 Jan 25;17(2):477-98; or Selection on the codon bias of chloroplast and cyanelle genes in different plant and algal lineages, Morton BR, J Mol Evol. 1998 Apr;46(4):449-59.

The vector polynucleotide can be codon optimized for expression in a specific cell-type, tissue type, organ type, and/or subject type. In some aspects, a codon optimized sequence is a sequence optimized for expression in a eukaryote, e.g., humans (i.e. being optimized for expression in a human or human cell), or for another eukaryote, such as another animal (e.g. a mammal or avian) as is described elsewhere herein. Such codon optimized sequences are within the ambit of the ordinary skilled artisan in view of the description herein. In some aspects, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.) , muscle cells (e.g. cardiac muscle, smooth muscle cells, and skeletal muscle cells), connective tissue cells ( fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some aspects, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some aspects, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some aspects, the vector is a non-viral vector or carrier. In some aspects, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with a heterologous DDP1 encoding polynucleotide and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid, polynucleotide molecule, or composition that be attached to or otherwise interact with, encapsulate, and/or associate with a polynucleotide to be delivered, such as a heterologous DDP1 encoding polynucleotide of the present invention.

### Naked Polynucleotides

In some aspects, one or more heterologous DDP1 encoding polynucleotides described elsewhere herein can be included in and/or delivered as a naked polynucleotide. The term of art “naked polynucleotide” as used herein refers to polynucleotides that are not associated with another molecule (e.g. proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the heterologous DDP1 encoding polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g. plasmids and artificial chromosomes), molecules that contain portions that are single stranded and portions that are double stranded (e.g. ribozymes), and the like. In some aspects, the naked polynucleotide contains only the heterologous DDP1 encoding polynucleotide(s) described herein. In some aspects, the naked polynucleotide can contain other nucleic acids and/or polynucleotides in addition to the heterologous DDP1 encoding polynucleotide(s) described herein. The naked polynucleotides can include one or more elements of a transposon system. Transposons and system thereof are described in greater detail elsewhere herein.

### Non-Viral Polynucleotide Vectors

In some aspects, one or more of the heterologous DDP1 encoding polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, agrobacterium vectors (Ti or Ri vectors), PSK systems (post-segregationally killing systems), ORT (operator repressor titration) plasmids, and the like. See e.g. Hardee et al. 2017. Genes. 8(2):65.

In some aspects, the non-viral polynucleotide vector can have a conditional origin of replication. In some aspects, the non-viral polynucleotide vector can be an ORT plasmid. In some aspects, the non-viral polynucleotide vector can have a minimalistic immunologically defined gene expression. In some aspects, the non-viral polynucleotide vector can have one or more post-segregationally killing system genes. In some aspects, the non-viral polynucleotide vector is AR-free. In some aspects, the non-viral polynucleotide vector is a minivector. In some aspects, the non-viral polynucleotide vector includes a nuclear localization signal. In some aspects, the non-viral polynucleotide vector can include one or more CpG motifs. In some aspects, the non-viral polynucleotide vectors can include one or more scaffold/matrix attachment regions (S/MARs). See e.g. Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In aspects, the S/MAR sequence is located downstream of an actively transcribed polynucleotide (e.g. one or more heterologous DDP1 encoding polynucleotides described herein) included in the non-viral polynucleotide vector. In some aspects, the S/MAR can be a S/MAR from the beta-interferon gene cluster. See e.g. Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some aspects, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some aspects, the non-viral polynucleotide vector can be a retrotransposon vector. In some aspects, the retrotransposon vector includes long terminal repeats. In some aspects, the retrotransposon vector does not include long terminal repeats. In some aspects, the non-viral polynucleotide vector can be a DNA transposon vector. DNA transposon vectors can include a polynucleotide sequence encoding a transposase. In some aspects, the transposon vector is configured as a non-autonomous transposon vector, meaning that the transposition does not occur spontaneously on its own. In some of these aspects, the transposon vector lacks one or more polynucleotide sequences encoding proteins required for transposition. In some aspects, the non-autonomous transposon vectors lack one or more Ac elements.

In some aspects a non-viral polynucleotide transposon vector system can include a first polynucleotide vector that contains the heterologous DDP1 encoding polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g. the heterologous DDP1 encoding polynucleotide(s)) and integrate it into one or more positions in the host cell’s genome. In some aspects the transposon vector or system thereof can be configured as a gene trap. In some aspects, the TIRs can be configured to flank a strong splice acceptor site followed by a reporter and/or other gene (e.g. one or more of the heterologous DDP1 encoding polynucleotide(s)) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, Sleeping Beauty transposon system (Tcl/mariner superfamily) (see e.g. Ivics et al. 1997. Cell. 91(4): 501-510), piggyBac (piggyBac superfamily) (see e.g. Li et al. 2013 110(25): E2279-E2287 and Yusa et al. 2011. PNAS. 108(4): 1531-1536), Tol2 (superfamily hAT), Frog Prince (Tcl/mariner superfamily) (see e.g. Miskey et al. 2003 Nucleic Acid Res. 31(23):6873-6881) and variants thereof.

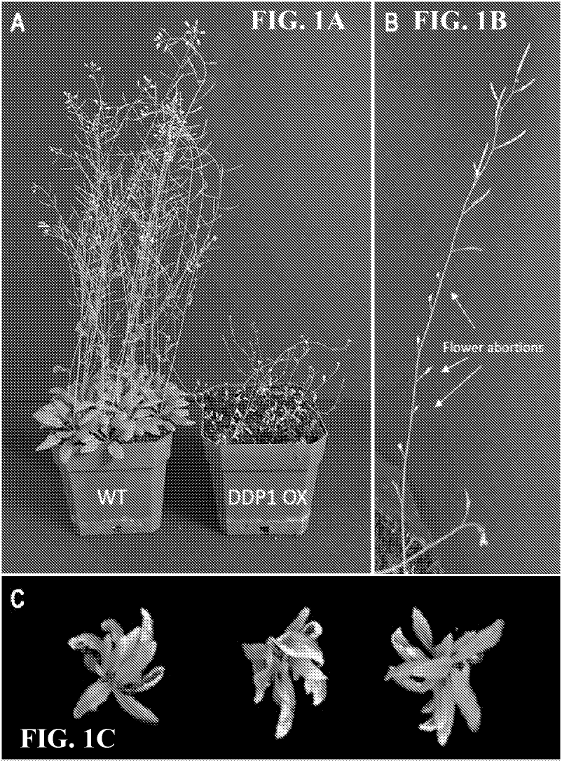

In some embodiments, the non-viral vector or vector system is an agrobacterium vector or vector system .In some embodiemnts the heterologous DDP1 encoding polynucleotide(s) is included in a T-DNA (or Ti) vector or an Ri vector (See e.g., Gelvin, S. 2003. Microbiol Mol Biol Rev. 2003 Mar; 67(1): 16-37, particularly at FIG. 1A; Lee and Gelvin. Plant Physiol. 2008 Feb; 146(2): 325-332 and as described elsewhere herein.

### Chemical Carriers

In some aspects, the heterologous DDP1 encoding polynucleotide(s) can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the heterologous DDP1 encoding polynucleotide(s)), (2) those capable of targeting specific cells, (3) those capable of increasing delivery of the polynucleotide (such as the heterologous DDP1 encoding polynucleotide(s)) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the heterologous DDP1 encoding polynucleotide(s) described herein. Suitable sizes include macro-, micro-, and nano-sized particles.

In some aspects, the non-viral carrier can be an inorganic particle. In some aspects, the inorganic particle, can be a nanoparticle. The inorganic particles can be configured and optimized by varying size, shape, and/or porosity. In some aspects, the inorganic particles are optimized to escape from the reticulo endothelial system. In some aspects, the inorganic particles can be optimized to protect an entrapped molecule from degradation., the Suitable inorganic particles that can be used as non-viral carriers in this context can include, but are not limited to, calcium phosphate, silica, metals (e.g. gold, platinum, silver, palladium, rhodium, osmium, iridium, ruthenium, mercury, copper, rhenium, titanium, niobium, tantalum, and combinations thereof), magnetic compounds, poarticles, and materials, (e.g. supermagnetic iron oxide and magnetite), quantum dots, fullerenes (e.g. carbon nanoparticles, nanotubes, nanostrings, and the like), and combinations thereof. Other suitable inorganic non-viral carriers are discussed elsewhere herein.

In some aspects, the non-viral carrier can be lipid-based. Suitable lipid-based carriers are also described in greater detail herein. In some aspects, the lipid-based carrier includes a cationic lipid or an amphiphilic lipid that is capable of binding or otherwise interacting with a negative charge on the polynucleotide to be delivered (e.g. such as an heterologous DDP1 encoding polynucleotide). In some aspects, chemical non-viral carrier systems can include a polynucleotide such as the heterologous DDP1 encoding polynucleotide (s)) and a lipid (such as a cationic lipid). These are also referred to in the art as lipoplexes. Other aspects of lipoplexes are described elsewhere herein. In some aspects, the non-viral lipid-based carrier can be a lipid nano emulsion. Lipid nano emulsions can be formed by the dispersion of an immisicible liquid in another stabilized emulsifying agent and can have particles of about 200 nm that are composed of the lipid, water, and surfactant that can contain the polynucleotide to be delivered (e.g. the heterologous DDP1 encoding polynucleotide(s)). In some aspects, the lipid-based non-viral carrier can be a solid lipid particle or nanoparticle.

In some aspects, the non-viral carrier can be peptide-based. In some aspects, the peptide-based non-viral carrier can include one or more cationic amino acids. In some aspects, 35 to 40, 45, 50, 55, 60, 65, 70, 75, 80, 85, 90, 95, 99 or 100% of the amino acids are cationic. In some aspects, peptide carriers can be used in conjunction with other types of carriers (e.g. polymer-based carriers and lipid-based carriers to functionalize these carriers). In some aspects, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g. US Pat. Pub. 2017/0079916 whose techniques and compositions can be adapted for use with the heterologous DDP1 encoding polynucleotide(s)), polymethacrylate, and combinations thereof.

In some aspects, the non-viral carrier can be configured to release an engineered delivery system polynucleotide that is associated with or attached to the non-viral carrier in response to an external stimulus, such as pH, temperature, osmolarity, concentration of a specific molecule or composition (e.g. calcium, NaCl, and the like), pressure and the like. In some aspects, the non-viral carrier can be a particle that is configured includes one or more of the heterologous DDP1 encoding polynucleotide(s) described herein and an environmental triggering agent response element, and optionally a triggering agent. In some aspects, the particle can include a polymer that can be selected from the group of polymethacrylates and polyacrylates. In some aspects, the non-viral particle can include one or more aspects of the compositions microparticles described in US Pat. Pubs. 20150232883 and 20050123596, whose techniques and compositions can be adapted for use in the present invention.

In some aspects, the non-viral carrier can be a polymer-based carrier. In some aspects, the polymer is cationic or is predominantly cationic such that it can interact in a charge-dependent manner with the negatively charged polynucleotide to be delivered (such as the heterologous DDP1 encoding polynucleotide(s)).

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a heterologous DDP1 encoding polynucleotide, into a virus particle and producing said virus particle when used alone or with one or more other viral vectors (such as in a viral vector system). Viral vectors and systems thereof can be used for producing viral particles for delivery of and/or expression of one or more components of the heterologous DDP1 encoding polynucleotide described herein. The viral vector can be part of a viral vector system involving multiple vectors. In some aspects, systems incorporating multiple viral vectors can increase the safety of these systems. Suitable viral vectors can include any plant viral vector or system such as any of those set forth in e.g., K. Hefferon. Biomedicines. Zaidi* and Mansoor. 2017 Sep; 5(3): 44, Front. Plant Sci., 11 Apr. 2017. https://doi.org/10.3389/fpls.2017.00539, and Abrahamian et al. 2020. Ann. Rev. Virol. 7:513-535, particularly those based on tobacco mosaic virus (TMV), Potexviruses, and Comovirus Cowpea mosaic virus (CPMV). Other aspects of viral vectors and viral particles produce therefrom are described elsewhere herein. In some aspects, the viral vectors are configured to produce replication incompetent viral particles for improved safety of these systems.

### Vector Construction

The vectors described herein can be constructed using any suitable process or technique. In some aspects, one or more suitable recombination and/or cloning methods or techniques can be used to the vector(s) described herein. Suitable recombination and/or cloning techniques and/or methods can include, but not limited to, those described in U.S. Application Publication No. US 2004-0171156 A1. Other suitable methods and techniques are described elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

### Virus Particle Production From Viral Vectors

In some aspects, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some aspects, the suitable host cell for virus production from viral vectors and systems thereof described herein can stably express one or more genes involved in packaging (e.g. pol, gag, and/or VSV-G) and/or other supporting genes.

In some aspects, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g. a heterologous DDP1 encoding polynucletoide), and virus particle assembly, and secretion of mature virus particles into the culture media. Various other methods and techniques are generally known to those of ordinary skill in the art.

Mature virus particles can be collected from the culture media by a suitable method. In some aspects, this can involve centrifugation to concentrate the virus. The titer of the composition containing the collected virus particles can be obtained using a suitable method. Such methods can include transducing a suitable cell line (e.g. NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some aspects, the resulting composition containing virus particles can contain 1 ×10¹ -1 × 10²⁰ particles/mL.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., heterologous DDP1 transcripts, proteins, enzymes, mutant forms thereof, fusion proteins thereof, etc.), and virus particles (such as from viral vectors and systems thereof).

One or more heterologous DDP1 encoding polynucleotides can be delivered using an engineered plant virus particle containing the one or more heterologous DDP1 encoding polynucleotides using appropriate formulations and doses.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell’s biological processes or biological characteristics to facilitate transport of the vector (with or without a carrier) into a cell. For example, the vector and/or its carrier can stimulate an endocytosis or similar process in the cell to facilitate uptake of the vector into the cell.

### Engineered DDP-1 Overexpressing Cells and Plants

Described herein are engineered cells, cell populations, and organisms that can be modified by any suitable polynucleotide and/or genome modifying agent(s) and/or systems described herein or that are generally known to one of ordinary skill in the art to contain and/or express a heterologous DDP1 encoding polynucleotide, vector or vector system, and/or heterologous DDP1 polypeptide. The engineered cells, cell populations, and organisms can have an insertion of one or more polynucleotides (e.g., a DDP1 encoding polynucleotide) and optionally insertion, deletion of one or more polynucleotides, mutation of one or more other polynucleotides, or a combination thereof. Cells, including cells in an organism, can be modified in vitro, in situ, ex vivo, or in vivo. In some embodiments, the modification is insertion or deletion of a polynucleotide, gene, or allele of interest. In some embodiments, the engineered cells are plant cells. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants. The term plant also encompasses offspring, cuttings, grafts, and the like.

Also provided herein are engineered plants that contain and/or express a heterologous DDP1 encoding polynucleotide, vector or vector system, and/or heterologous DDP1 polypeptide in one or more cells. Also provided herein are engineered plants that contain and/or express a heterologous DDP1 encoding polynucleotide, vector or vector system, and/or heterologous DDP1 polypeptide in all cells. Also provided herein are engineered plants that contain and/or express a heterologous DDP1 encoding polynucleotide, vector or vector system, and/or heterologous DDP1 polypeptide in some cells.

In some embodiments, a cell and/or plant expressing a heterologous DDP1 as described herein can accumulate or sequester 1-100 (e.g., 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100) times more Pi from its environment, such as a soil or water source, as compared to a wild-type and/or non-DDP1 expressing control plant. In some embodiments, a cell and/or plant expressing a heterologous DDP1 as described herein can accumulate or sequester 1-10 (e.g., 1, to/or 2, 3, 4, 5, 6, 7, 8, 9, 10) times more Pi from its environment, such as a soil or water source, as compared to a wild-type and/or non-DDP1 expressing control plant. In some embodiments, a cell and/or plant expressing a heterologous DDP1 as described herein can accumulate or sequester 1-6 (e.g., 1, to/or 2, 3, 4, 5, 6,) times more Pi from its environment, such as a soil or water source, as compared to a wild-type and/or non-DDP 1 expressing control plant. In some embodiments, a cell and/or plant expressing a heterologous DDP1 as described herein can accumulate or sequester 1-5 (e.g., 1, to/or 2, 3, 4, 5) times more Pi from its environment, such as a soil or water source, as compared to a wild-type and/or non-DDP1 expressing control plant. In some embodiments, a cell and/or plant expressing a heterologous DDP1 as described herein can accumulate or sequester 1-2 (e.g., 1, to/or 2) times more Pi from its environment, such as a soil or water source, as compared to a wild-type and/or non-DDP1 expressing control plant.

Also described herein are engineered plants that can contain one or more of the engineered cells expressing a heterologous DDP1 described herein. In some embodiments, the engineered plant is an engineered monocotyledonous plant or an engineered dicotyledonous plant. In some embodiments the engineered dicotyledonous plant belongs to the order Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Brassicales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, Brassicales, or Asterales.

In some embodiments, the engineered monocotyledonous plant belongs to the order Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, Orchidales, Pinales, Ginkogoales, Cycadales, Araucariales, Cupressales or Gnetales.

In some embodiments, the engineered plant is a species of Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Thlaspi, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, or Pseudotsuga.

In some embodiments, the engineered plant is a fern. In some embodiments, the engineered plant is a moss. In some embodiments, the engineered plant is a liverwort. In some embodiments, the engineered plant is of the phyla Bryophyta, Pterophyta, or Lycopodiophita. In some embodiments, the engineered plant is from the order Sphagnales, Jungermanniales, Marchantiles, Polytrichales, Hypnobryales, Dicranales, Polypodiales, Equisetales, or Selaginellales. In some embodiments, the engineered plant is from the genus Sphagnum, Bazzania, Marchantia, Conocephalum, Riccia, Polytrichum, Pleurozium, Dicranum, Nephrolepis, Equistum, or Selaginella. In some embodiments, the engineered plant is Marchantia polymorpha.

In some embodiments, the engineered plant is a grain crop plant (e.g., wheat, maize, rice, millet, barley), a fruit crop plant (e.g., tomato, apple, pear, strawberry, orange), forage crops (e.g., alfalfa), a root vegetable crop plant (e.g., carrot, potato, sugar beets, yam), a leafy vegetable crop plant (e.g., lettuce, spinach); a flowering crop plant (e.g., petunia, rose, chrysanthemum), a conifers or pine tree (e.g., pine fir, spruce); a plant used in phytoremediation (e.g., heavy metal accumulating plants); an oil crop plant(e.g., sunflower, rape seed), or a plant typically used for experimental purposes (e.g., Arabidopsis).

In some embodiments, the engineered plant is an angiosperm or a gymnosperm plant.

In some embodiments, the engineered plant is acacia, alfalfa, amaranth, apple, apricot, artichoke, ash tree, asparagus, avocado, banana, barley, beans, beet, birch, beech, blackberry, blueberry, broccoli, Brussel’s sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pennycress, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, or zucchini.

In some embodiments, the engineered plant is a turfgrass. In some embodiments, the engineered plant is of the order Cyperales. In some embodiments, the engineered plant is from the family of Poaceae. In some embodiments, the engineered plant is of the genus Achnatherum P. Beauv. (needlegrass), Achnella Barkworth (ricegrass), Acrachne Chiov. (goosegrass), Acroceras Stapf (acroceras), Aegilops L. (goatgrass), Aegopogon Humb. & Bonpl. ex Willd. (relaxgrass), Aeluropus Trin. (Indian walnut), ×Agroelymus E.G. Camus ex A. Camus (agroelymus), ×Agropogon Fourn. (agropogon), Agropyron Gaertn. (wheatgrass), Agrostis L. (bentgrass), Aira L. (hairgrass), Allolepis Söderst. & Decker (Texas salt), Alloteropsis J. Presl (summergrass), Alopecurus L. (foxtail), Amblyopyrum Eig (amblyopyrum), ×Ammocalamagrostis P. Fourn., Ammophila Host (beachgrass), Ampelodesmos Link (Mauritanian grass), Amphibromus Nees (wallaby grass), Amphicarpum Kunth (maidencane), Ancistrachne S.T. Blake, Andropogon L. (bluestem), Anthaenantia P. Beauv. (silkyscale), Anthephora Schreb. (oldfield grass), Anthoxanthum L. (homwort), Apera Adans. (silkybent), Apluda L. (Mauritian grass), Arctagrostis Griseb. (polargrass), ×Arctodupontia Tzvelev (arctodupontia), Arctophila Rupr. ex Andersson (pendantgrass ), Genus Aristida L. (threeawn), Arrhenatherum P. Beauv. (oatgrass), Arthraxon P. Beauv. (carpetgrass), Arthrostylidium Rupr. (climbing bamboo), Arundinaria Michx. (cane), Arundinella Raddi (rabo de gato), Arundo L. (giant reed), Astrebla F. Muell. ex Benth., Austrostipa S.W.L. Jacobs & J. Everett, Avellinia Parl., Avena L. (oat), Avenula (Dumort.) Dumort. (oatgrass), Axonopus P. Beauv. (carpetgrass), Bambusa Schreb. (bamboo), Beckmannia Host (sloughgrass), Blepharidachne Hack. (desertgrass), Blepharoneuron Nash (dropseed), Borinda Stapleton (borinda), Bothriochloa Kuntze (beardgrass), Bouteloua Lag. (grama), Brachiaria (Trin.) Griseb. (signalgrass),Brachyachne Stapf, Brachyelytrum P. Beauv. (shorthusk), Brachypodium P. Beauv. (false brome), Brachypodium distachyon, Briza L. (quakinggrass), Bromidium Nees & Meyen (tropical bent), Bromus L. (brome), Calamagrostis Adans. (reedgrass), ×Calammophila Brand (calammophila), Calamovilfa (A. Gray) Hack. ex Scribn. & Southworth (sandreed), Calyptochloa C.E. Hubb., Castellia Tineo, Catabrosa P. Beauv. (whorlgrass), Catapodium Link (femgrass), Cathestecum J. Presl (false grama), Celtica F.M. Vazquez & Barkworth, Cenchrus L. (sandbur), Centotheca Desv., Centropodia (R. Br.) Rchb., Chasmanthium Link (woodoats), Chloris Sw. (windmill grass), Chrysopogon Trin. (false beardgrass), Chusquea Kunth (chusquea bamboo), Cinna L. (woodreed), Cladoraphis Franch. (bristly lovegrass), Cleistachne Benth., Cleistogenes Keng, Coelorachis Brongn. (jointtail grass), Coix L. (Job’s tears), Coleanthus Seidel (mossgrass), Cortaderia Stapf (pampas grass), Corynephorus P. Beauv. (clubawn grass), Cottea Kunth (cotta grass), Crypsis Aiton (pricklegrass), Ctenium Panzer (toothache grass), Cutandia Willk. (Memphisgrass), Cymbopogon Spreng. (lemon grass), Cynodon Rich. (Bermudagrass), Cynosurus L . (dogstail grass), Cyrtococcum Stapf, Dactylis L. (orchardgrass), Dactyloctenium Willd. (crowfoot grass), Danthonia DC. (oatgrass), Danthoniopsis Stapf, Dasyochloa Willd. ex Rydb. (woollygrass), Dasypyrum (Coss. & Durieu) T. Dur. (mosquitograss), Dendrocalamus Nees, Deschampsia P. Beauv. (hairgrass), Desmostachya (Stapf) Stapf, Diarrhena P. Beauv. (beakgrain), Dichanthelium (Hitchc. & Chase) Gould (rosette grass), Dichanthium Willem. (bluestem), Dichelachne Endl. (Plumegrass), Diectomis Kunth (foldedleaf grass ), Digitaria Haller (crabgrass), Dimeria R. Br., Dinebra Jacq. (viper grass), Dissanthelium Trin. (Catalina grass), Dissochondrus (Hillebr.) Kuntze (false brittlegrass), Distichlis Raf. (saltgrass), ×Dupoa J. Cay. & S.J. Darbyshire (dupoa), Dupontia R. Br. (tundragrass), Echinochloa P. Beauv. (cockspur grass), Echinopogon P. Beauv., Ectrosia R. Br. (ectrosia), Ehrharta Thunb. (veldtgrass), Eleusine Gaertn. (goosegrass), Elionurus Humb. & Bonpl. ex Willd. (balsamscale grass), ×Elyhordeum Mansf. ex Zizin & Petrowa (barley), ×Elyleymus Baum (wildrye), Elymus L . (wildrye), Elytrigia Desv., Enneapogon Desv. ex P. Beauv. (feather pappusgrass), Enteropogon Nees (umbrellagrass), Entolasia Stapf (entolasia) Eragrostis von Wolf (lovegrass), Eremochloa Büse (centipede grass), Eremopyrum (Ledeb.) Jaubert & Spach (false wheatgrass), Eriachne R. Br., Eriochloa Kunth (cupgrass), Eriochrysis P. Beauv. (moco de pavo), Erioneuron Nash (woollygrass), Euclasta Franch. (mock bluestem), Eulalia Trin., Eulaliopsis Honda (sabaigrass), Eustachys Desv. (fingergrass), Festuca L. (fescue), ×Festulolium Asch. & Graebn. (Festulolium), Fingerhuthia Nees (Zulu fescue), Garnotia Brongn. (lawngrass), Gastridium P. Beauv. (nit grass), Gaudinia P. Beauv. (fragile oat), Gigantochloa Kurz ex Munro (gigantochloa), Glyceria R. Br. (mannagrass), Gymnopogon P. Beauv. (skeletongrass), Gynerium Willd. ex P. Beauv. (wildcane), Hackelochloa Kuntze (pitscale grass), Hainardia Greuter (barbgrass), Hakonechloa Makino ex Honda (Hakone grass), Helictotrichon Besser ex Schult. & Schult. f. (alpine oatgrass), Hemarthria R. Br. (jointgrass), Hesperostipa (Elias) Barkworth (needle and thread), Heteranthelium Hochst. ex Jaub. & Spach, Heteropogon Pers. (tanglehead), Hierochloe R. Br. (sweetgrass), Hilaria Kunth (curly-mesquite), Holcus L. (velvetgrass), Homolepis Chase (panicgrass), Homopholis C.E. Hubb., Hordelymus (Jess.) Harz, Hordeum L. (barley), Hygroryza Nees (watergrass), Hymenachne P. Beauv. (marsh grass), Hyparrhenia Andersson ex Fourn. (thatching grass), Hypogynium Nees (West Indian bluestem), Ichnanthus P. Beauv. (bedgrass), Imperata Cirillo (satintail), Isachne R. Br. (bloodgrass), Ischaemum L. (murainagrass), Iseilema Andersson, Ixophorus Schltdl. (Central America grass), Jarava Ruiz & Pav. (rice grass), Kalinia H.L. Bell & Columbus (kalinia grass), Karroochloa Conert & Türpe (South African oatgrass), Koeleria Pers. (Junegrass), Lagurus L. (harestail grass), Lamarckia Moench (goldentop grass), Lasiacis (Griseb.) Hitchc. (smallcane), Lasiurus Boiss., Leersia Sw. (cutgrass), Leptochloa P. Beauv. (sprangletop), Leptochloopsis Yates (limestone grass), Leptocoryphium Nees (lanilla), Lepturus R. Br. (thintail), Leucopoa Griseb. (spike fescue), ×Leydeum Barkworth (hybrid ryegrass), Leymus Hochst. (wildrye), Limnodea L.H. Dewey (Ozark grass), Lithachne P. Beauv. (diente de perro), Loliolum Krecz. & Bobr., Lolium L. (ryegrass), Lophatherum Brongn. (lophatherum), Loudetia Hochst., Luziola Juss. (watergrass), Lycurus Kunth -(wolfstail), Lygeum Loefl. ex L. (lygeum), Melica L. (melicgrass), Melinis P. Beauv. (stinkgrass), Merxmuellera Conert, Mibora Adans. (sandgrass), Microchloa R. Br. (smallgrass), Microlaena R. Br. (weeping grass), Micropyrum Link, Microstegium Nees (browntop), Milium L. (milletgrass), Miscanthus Andersson (silvergrass), Mnesithea Kunth (jointtail grass), Molinia Schrank (moorgrass), Monanthochloe Engelm. (shoregrass), Muhlenbergia Schreb. (muhly), Munroa Torr., orth. cons. (false buffalograss), Nardus L. (matgrass), Nassella (Trin.) Desv. (needlegrass), Neeragrostis Bush (creeping lovegrass), Neololeba Widjaja (neololeba), Neostapfia Burtt Davy (Colusagrass), Neyraudia Hook. f. (neyraudia), Ochthochloa Edgew., Olyra L. (carrycillo), Ophiuros C.F. Gaertn., Oplismenus P. Beauv. (basketgrass), Orcuttia Vasey (Orcutt grass), Oryza L. (rice), Oryzopsis Michx. (ricegrass), Ottochloa Dandy, Oxychloris M. Lazarides, Oxytenanthera Munro (oxytenanthera), Panicum L. (panicgrass), Pappophorum Schreb. (pappusgrass), Parapholis C.E. Hubbard (sicklegrass), Pascopyrum A. Löve (wheatgrass), Paspalidium Stapf (watercrown grass), Paspalum L. (crowngrass), Patis Ohwi (ricegrass), Pennisetum Rich. ex Pers. (fountaingrass), Perotis Aiton, Phalaris L. (canarygrass), Phanopyrum (Raf.) Nash (savannah-panicgrass), Pharus L. (stalkgrass), Phippsia (Trin.) R. Br. (icegrass), Phleum L. (timothy), Phragmites Adans. (reed), Phyllostachys Siebold & Zucc. (bamboo), Piptatheropsis Romasch., P.M. Peterson & R. J. Soreng (ricegrass), Piptatherum P. Beauv. (ricegrass), Piptochaetium J. Presl (speargrass), Plectrachne Henrard, Pleioblastus Nakai (dwarf bamboo), Pleuraphis Torr. (galleta grass), Pleuropogon R. Br. (semaphoregrass), Poa L. (bluegrass), Pogonatherum P. Beauv., Polypogon Desf. (rabbitsfoot grass), Polytoca R. Br., Polytrias Hack. (Java grass), Psathyrostachys Nevski (wildrye), ×Pseudelymus Barkworth & D.R. Dewey (foxtail wheatgrass), Pseudoroegneria (Nevski) A. Löve (wheatgrass), Pseudosasa Makino ex Nakai (arrow bamboo), Psilurus Trin., Ptilagrostis Griseb. (false needlegrass), Puccinellia Parl. (alkaligrass), ×Pucciphippsia Tzvelev (pucciphippsia), Redfieldia Vasey (blowout grass), Reimarochloa Hitchc. (reimar grass), Rostraria Trin. (hairgrass), Rottboellia L. f. (itchgrass), Rytidosperma Steud. (wallaby grass), Saccharum L. (sugarcane), Sacciolepis Nash (cupscale grass), Sasa Makino & Shib. (broadleaf bamboo), ×Schedolium Holub (fescue ryegrass), Schedonnardus Steud. (tumblegrass), Schedonorus P. Beauv. (fescue), Schismus P. Beauv. (Mediterranean grass), Schizachne Hack. (false melic), Schizachyrium Nees (little bluestem), Schizostachyum Nees (Polynesian ‘ohe P), Schmidtia Moench, Sclerochloa P. Beauv. (hardgrass), Scleropogon Phil. (burrograss), Sclerostachya A. Camus, Scolochloa Link (rivergrass), Scribneria Hack. (Scribner’s grass), Secale L. (rye), Sehima Forssk., Genus Semiarundinaria Makino, Sesleria Scop., Setaria P. Beauv. (bristlegrass), Setaria viridis (e.g. of the Andropogoneae tribe), Setariopsis Scribn. ex Millsp. (setariopsis), Shibataea Makino ex Nakai, Sinocalamus McClure (wideleaf bamboo), Snowdenia C.E. Hubb., Sorghastrum Nash (Indiangrass), Sorghum Moench (sorghum), Spartina Schreb. (cordgrass), Sphenopholis Scribn. (wedgescale), Spinifex L., Spodiopogon Trin., Sporobolus R. Br. (dropseed), Steinchisma Raf. (gaping grass), Stenotaphrum Trin. (St. Augustine grass), Stipa L., Stipagrostis Nees, Swallenia Söderst. & Decker (dunegrass), Taeniatherum Nevski (medusahead), Tetrachne Nees, Tetrapogon Desf., Thaumastochloa C.E. Hubb., Themeda Forssk. (kangaroo grass), Thinopyrum A. Löve (wheatgrass), Thuarea Pers. (Kuroiwa grass), Thyridolepis S.T. Blake, Thysanolaena Nees (tiger grass), Torreyochloa Church (false mannagrass), Trachypogon Nees (crinkleawn grass), Tragus Haller (bur grass), Tribolium Desv. (tribolium), Trichloris Fourn. ex Benth. (false Rhodes grass), Tricholaena Schrad., Trichoneura Andersson (Silveus’ grass), Tridens Roem. & Schult. (tridens), Triodia R. Br., Triplasis P. Beauv. (sandgrass), Tripogon Roem. & Schult. (fiveminute grass), Tripsacum L. (gamagrass), Triraphis R. Br. (needlegrass), Trisetum Pers. (oatgrass), ×Triticosecale Wittm. ex A. Camus (triticale), Triticum L . (wheat), Tuctoria J. Reeder (spiralgrass), Uniola L. (seaoats), Urochloa P. Beauv. (signalgrass), Vahlodea Fr. (hairgrass), Vaseyochloa Hitchc. (Texasgrass), Ventenata Koeler (North Africa grass), Vetiveria Bory (vetivergrass), Vossia Wall. & Griffith (hippo grass), Vulpia C.C. Gmel. (fescue), Whiteochloa C.E. Hubb., Willkommia Hack. (willkommia), Zea L. (corn), Zingeria P.A. Smirn., Zizania L. (wildrice), Zizaniopsis Döll & Asch. (cutgrass), P. australis, or Zoysia Willd. (lawngrass).

In some embodiments, the engineered plant is of the genus Striga. In some embodiments, the engineered plant is witchweed (e.g. Striga hermonthica).

In some embodiments, the engineered plant is of the genus Cardamine. In some embodiments, the engineered plant is Cardamine hirsute (hairy bittercress).

In some embodiments, the engineered plant is an algae.

In some embodiments, the engineered plant is an algae from the phyla Rhodophyta (red algae), Chlorophyta (green algae), Phaeophyta (brown algae), Bacillariophyta (diatoms), Eustigmatophyta, a dinoflagellates, or the prokaryotic phylum Cyanobacteria (blue-green algae).

In some embodiments, the engineered algae the species of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, or Trichodesmium.

In some embodiments, the engineered cell, the engineered plant, or both include a DDP1 encoding polynucleotide stably integrated into the genome of the engineered cell.

In some embodiments, the engineered cell, the engineered plant, or both include a DDP1 encoding polynucleotide that is transiently expressed in the engineered cell, engineered plant, or both.

In some embodiments, the engineered cell, engineered cell population, and or engineered plant has one or more modulated observable traits as compared to an unmodified or control cell or plant. In some embodiments, the engineered plant has increased growth or performance in at least one economically important trait. In some embodiments, the engineered plant has substantially increased Pi accumulation and/or storage capacity.

### METHODS OF MAKING ENGINEERED PLANT CELLS AND PLANTS

Also described herein are methods of modifying a plant cell such that it contains and/or expresses a heterologous DDP1 polypeptide and/or encoding polynucleotide. In some embodiments, the method includes modifying a plant cell such that comprises a heterologous Diadenosine and Diphosphoinositol Polyphosphate Phosphohydrolase (DDP1) polypeptide, a heterologous DDP1 encoding polynucleotide, a vector or vector system containing a heterologous DDP1 encoding polynucleotide, or a combination thereof. In some embodiments, modifying includes delivering a polynucleotide having a sequence that is about 50-100% (e.g., 50, to/or 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%) identical to SEQ ID NO: 2 or a vector or vector system thereof to the cell, delivering a polypeptide having a sequence that is about 50-100% (e.g., 50, to/or 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%) identical to SEQ ID NO: 1 to the cell, or both.

In some embodiments, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

“Plant” as used herein encompasses any plant tissue or part of the plant of the invention. Preferably, said part is selected from the group consisting of a plant cell, a somatic embryo, a pollen, a gametophyte, an ovule, an inflorescence, a leaf, a seedling, a stem, a callus, a stolon, a microtuber, a shoot, a seed, a fruit and a spore. Further encompassed are T1 generation plants produced from the seeds of the transformed plant (T0). Any suitable method can be used to confirm and detect the modification made in the plant. Such methods are generally known in the art. In some examples, when a variety of modifications are made, one or more desired modifications or traits resulting from the modifications may be selected and detected. The detection and confirmation may be performed by biochemical and molecular biology techniques such as Southern analysis, PCR, Northern blot, S1 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

A part of a plant, e.g., a “plant tissue” can be engineered to include a DDP1 encoding polynucleotide, vector, and/or polypeptide described elsewhere herein to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized unites, such as, for example, plant tissue, a plant organ, or a whole plant.

A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processingcolored substances (e.g., the β-glucuronidase, luciferase, B or C1 genes).

Any suitable method may be used to deliver the transgene to the plant, plant cell, and/or plant cell population. Such transformation techniques are generally known in the art. Example methods and techniques include those in U.S. Pat. No. 6,603,061 - Agrobacterium-Mediated Plant Transformation Method; U.S. Pat. No. 7,868,149 - Plant Genome Sequences and Uses Thereof and US 2009/0100536 - Transgenic Plants with Enhanced Agronomic Traits, Morrell et al “Crop genomics: advances and applications,” Nat Rev Genet. 2011 Dec 29;13(2):85-96, all the contents and disclosure of each of which are herein incorporated by reference in their entirety.

In some embodiments, where transient expression is desired transgene DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

A method of generating engineered cells and/or plants can include transformation of one or more cells. The term “transformation” broadly refers to the process by which a plant host is genetically modified by the introduction of DNA by means of Agrobacteria or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, and shoots. A plant tissue also refers to any clone of such a plant, seed, progeny, propagule whether generated sexually or asexually, and descendants of any of these, such as cuttings or seed. The term “transformed” as used herein, refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is transmitted to the subsequent progeny. In these embodiments, the “transformed” or “transgenic” cell or plant may also include progeny of the cell or plant and progeny produced from a breeding program employing such a transformed plant as a parent in a cross and exhibiting an altered phenotype resulting from the presence of the introduced DNA molecule. Preferably, the transgenic plant is fertile and capable of transmitting the introduced DNA to progeny through sexual reproduction.

In some embodiments, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the DDP1 transgene is expressed. Vectors and vector systems are described in greater detail elsewhere herein.

The term plant also encompasses progeny of the plant The term “progeny”, such as the progeny of a transgenic (or engineered) plant, is one that is born of, begotten by, or derived from a plant or the transgenic plant. The introduced DNA molecule may also be transiently introduced into the recipient cell such that the introduced DNA molecule is not inherited by subsequent progeny and thus not considered “transgenic”. Accordingly, as used herein, a “non-transgenic” plant or plant cell is a plant which does not contain a foreign DNA stably integrated into its genome.

Also described herein are gametes, seeds, germplasm, embryos, either zygotic or somatic, progeny or hybrids of plants comprising the genetic modification (e.g., inclusion and/or expression of a DDP1 encoding polynucleotide), which are produced by traditional breeding methods, are also included within the scope of the present invention. Such plants may contain a heterologous or foreign DNA sequence inserted at or instead of a target sequence. Alternatively, such plants may contain only an alteration (mutation, deletion, insertion, substitution) in one or more nucleotides. As such, such plants will only be different from their progenitor plants by the presence of the particular modification.

### Stable Integration in the Genome of Plants and Plant Cells

In particular embodiments, the polynucleotides encoding the heterologous DDP1 are introduced for stable integration into the genome of a plant cell. In these embodiments, the design of the transformation vector or the expression system can be adjusted depending on for when, where and under what conditions the heterologous DDP1 encoding polynucleotides are expressed. Suitable vectors and delivery are described in greater detail elsewhere herein.

In particular embodiments, the heterologous DDP1 encoding polynucleotides are stably introduced into the genomic DNA of a plant cell. In particular embodiments, the heterologous DDP1 encoding polynucleotides are introduced for stable integration into the DNA of a plant organelle such as, but not limited to a plastid, mitochondrion or a chloroplast. In some embodiments, the expression system for stable integration into the genome of a plant cell can contain one or more of the following elements: a promoter element that can be used to express heterologous DDP1 encoding polynucleotide(s) in a plant cell; a 5′ untranslated region to enhance expression; an intron element to further enhance expression in certain cells, such as monocot cells; a multiple-cloning site to provide convenient restriction sites for inserting the polynucleotide modifying agent(s) or a system thereof and other desired elements; and a 3′ untranslated region to provide for efficient termination of the expressed transcript. The elements of the expression system may be on one or more expression constructs which are either circular such as a plasmid or transformation vector, or non-circular such as linear double stranded DNA.

DNA construct(s) containing the components of the systems, and, where applicable, template sequence may be introduced into the genome of a plant, plant part, or plant cell by a variety of conventional techniques. The process generally comprises the steps of selecting a suitable host cell or host tissue, introducing the construct(s) into the host cell or host tissue.

In particular embodiments, the DNA construct may be introduced into the plant cell using techniques such as but not limited to electroporation, microinjection, aerosol beam injection of plant cell protoplasts, or the DNA constructs can be introduced directly to plant tissue using biolistic methods, such as DNA particle bombardment (see also Fu et al., Transgenic Res. 2000 Feb;9(1): 11-9). The basis of particle bombardment is the acceleration of particles coated with gene/s of interest toward cells, resulting in the penetration of the protoplasm by the particles and typically stable integration into the genome. (see e.g. Klein et al, Nature (1987), Klein et ah, Bio/Technology (1992), Casas et ah, Proc. Natl. Acad. Sci. USA (1993).).

In particular embodiments, the DNA constructs containing components of the systems may be introduced into the plant by Agrobacterium-mediated transformation. The DNA constructs may be combined with suitable T-DNA flanking regions and introduced into a conventional Agrobacterium tumefaciens host vector. The foreign DNA can be incorporated into the genome of plants by infecting the plants or by incubating plant protoplasts with Agrobacterium bacteria, containing one or more Ti (tumor-inducing) plasmids. (see e.g. Fraley et al., (1985), Rogers et al., (1987) and U.S. Pat. No. 5,563,055).

### Transient Expression of in Plants and Plant Cells

In some embodiments, the heterologous DDP1 encoding polynucleotides can be transiently expressed in the plant cell. In these embodiments, the system can ensure DDP1 expression and any Pi accumulation can further be controlled. As the expression of the necessary components of the heterologous DDP1 encoding polynucleotide(s) is transient, plants regenerated from such plant cells typically contain no foreign DNA.

In particular embodiments, the heterologous DDP1 encoding polynucleotides can be transiently introduced in the plant cells using a plant viral vector (Scholthof et al. 1996, Annu Rev Phytopathol. 1996;34:299-323). In further particular embodiments, said viral vector is a vector from a DNA virus. For example, geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). In other particular embodiments, said viral vector is a vector from an RNA virus. For example, tobravirus (e.g., tobacco rattle virus, tobacco mosaic virus), potexvirus (e.g., potato virus X), or hordeivirus (e.g., barley stripe mosaic virus). The replicating genomes of plant viruses are non-integrative vectors. Other suitable vectors are described elsewhere herein

In particular embodiments, the vector used for transient expression of constructs in plants is for instance a pEAQ vector, which is tailored for Agrobacterium-mediated transient expression (Sainsbury F. et al., Plant Biotechnol J. 2009 Sep;7(7):682-93) in the protoplast. Precise targeting of genomic locations was demonstrated using a modified Cabbage Leaf Curl virus (CaLCuV) vector to express gRNAs in stable transgenic plants expressing a CRISPR enzyme (Scientific Reports 5, Article number: 14926 (2015), doi:10.1038/srep14926).

In particular embodiments, double-stranded DNA fragments encoding the heterologous DDP1 can be transiently introduced into the plant cell. In such embodiments, the introduced double-stranded DNA fragments are provided in sufficient quantity to modify the cell but do not persist after a contemplated period of time has passed or after one or more cell divisions. Methods for direct DNA transfer in plants are known by the skilled artisan (see for instance Davey et al. Plant Mol Biol. 1989 Sep;13(3):273-85.)

In other embodiments, an RNA polynucleotide encoding the heterologous DDP1 is introduced into the plant cell, which is then translated and processed by the host cell generating the protein in sufficient quantity accumulate Pi but which does not persist after a contemplated period of time has passed or after one or more cell divisions. Methods for introducing mRNA to plant protoplasts for transient expression are known by the skilled artisan (see for instance in Gallie, Plant Cell Reports (1993), 13;119-122).

In some embodiments, a combination of the different methods described above can be used.

### Translocation to and/or Expression in Specific Plant Organelles

The system may comprise elements for translocation to and/or expression in a specific plant organelle. In some embodiments, a tissue specific promoter can be included in the expression construct. In some embodiments, a tissue localization or organelle localization sequence or signal can be incorporated into the expression constructs. Such promoters and localization signals are described in greater detail elsewhere herein and/or will be appreciated by one of ordinary skill in the art.

### Chloroplast Targeting

In some embodiments, the engineered plants can be engineered to contain modified chloroplast genes or to ensure expression in the chloroplast. In some embodiments, chloroplast transformation methods or compartmentalization of the DDP1 encoding polynucleotides and/or polypeptides to the chloroplast. For instance, the introduction of genetic modifications in the plastid genome can reduce biosafety issues such as gene flow through pollen.

Methods of chloroplast transformation are known in the art and include Particle bombardment, PEG treatment, and microinjection. Additionally, methods involving the translocation of transformation cassettes from the nuclear genome to the plastid can be used as described in WO2010061186.

In some embodiments, one or more of the heterologous DDP1 encoding polynucleotides can be targeted to the plant chloroplast. This can be achieved by incorporating in the expression construct a sequence encoding a chloroplast transit peptide (CTP) or plastid transit peptide, operably linked to the 5′ region of the sequence encoding the Cas protein. The CTP is removed in a processing step during translocation into the chloroplast. Chloroplast targeting of expressed proteins is well known to the skilled artisan (see for instance Protein Transport into Chloroplasts, 2010, Annual Review of Plant Biology,Vol. 61: 157-180). In such embodiments it is also can be desirable to target the guide RNA to the plant chloroplast. Methods and constructs which can be used for translocating guide RNA into the chloroplast by means of a chloroplast localization sequence are described, for instance, in US 20040142476, incorporated herein by reference. Such variations of constructs can be incorporated into the expression systems of the invention to efficiently translocate the heterologous DDP1 encoding polynucleotides.

### METHODS OF USING THE ENGINEERED PLANTS

The engineered plants described herein can be propagated, grown, harvested, and/or cultivated for any purpose such as food or commodity production and/or fertilizer. In some embodiments, a method can include cultivating a plant as described elsewhere herein.

### Methods of Soil Remediation

The engineered plants described herein can be propagated, grown, and/or cultivated for soil remediation. As described and demonstrated elsewhere herein, the DDP1 overexpressing plants can be capable of sequestering Pi from the soil and accumulating it in greater amounts as compared to a non-DDP1 expressing control plant (or wild-type) plant. Thus, in this way, growth of a DDP1 expressing plant can facilitate reducing soil levels of Pi. In some embodiments, the DDP1 expressing plant is grown in a soil that has a high level of phosphate. In some embodiments, the DDP1 expressing plant has DDP1 under control of an inducible promoter. In some embodiments, DDP1 expression is not induced until after the DDP1 expressing plant has reached a desired growth stage, util after a fruit or other component of the plant has been harvested, and/or until after a set period of time after planting, germinating, and/or sprouting.

In some embodiments, DDP1 expression is not induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days post planting, germinating, and/or sprouting. In some embodiments, DDP1 expression is not induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks post planting, germinating, and/or sprouting.. In some embodiments, DDP1 expression is not induced for 1, 2, 3, 4, 5, or more years post planting, germinating, and/or sprouting.

In some embodiments, DDP1 expression is induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, DDP1 expression is induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks. In some embodiments, DDP1 expression is induced for1, 2, 3, 4, 5, or more years.

### Products Produced From the DDP1 Overexpressing Plants

In some embodiments, the DDP1 overexpressing plants and/or parts (e.g., fruits, nuts, seeds, grains, etc. ) are harvested. In some embodiments, one or more parts or the whole plant is harvested for fertilizer. In some embodiments, after one or more parts of the DDP1 expressing plant is harvested for a non-fertilizer product, the rest is harvested for fertilizer production. Once a DDP1 expressing plant has accumulated the Pi, it can be harvested and be used in any suitable form as a fertilizer. In this way, sequestered Pi can be reapplied when desired as a fertilizer and thus serve to recycle phosphorous.

In some embodiments, the harvested DDP1 expressing plants can be applied as a fresh-biomass fertilizer. Such fresh biomass can be chopped, shredded, pulverized, and/or otherwise mechanically broken down prior to application.

In other embodiments, the harvested DDP1 expressing plants can be further processed, such as carbonized, prior to applying as a fertilizer. In some embodiments, the harvested DDP1 plants can be processed into biochar. “Biochar” as used herein is a term of art and refers to a charcoal-like byproduct of the process of pyrolysis, or the anaerobic (meaning without oxygen) thermal decomposition of organic material, particularly plant material in the present context. The biochar can be applied as a fertilizer for plant cultivation.

The cycle can be continued by growing DDP1 plants in soil fertilized with DPP1 fresh biomass and/or biochar.

Thus, also described herein is biochar produced from DDP1 expressing plants that have accumulated/sequestered Pi from the soil in which they were grown. In some embodiments, the biochar has a slower phosphorus release rate than biochar produced from a wild-type plant (or non-DDP1 expressing control plant). In some embodiments, the biochar produced from DDP1 expressing plants that have accumulated/sequestered Pi from the soil in which they were grown releases phosphorous at a rate 1-1000 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 times slower than biochar produced from a wild-type plant (or non-DDP1 expressing control plant). In some embodiments, the biochar produced from DDP1 expressing plants that have accumulated/sequestered Pi from the soil in which they were grown releases phosphorous about twice as slow (e.g. 50% of the rate) as biochar produced from a wild-type plant (or non-DDP1 expressing control plant).

In some embodiments, the biochar is combined with one or more other nutrients, soil fortifiers, soils, mulches, binders, or other soil additives.

### Methods of Fertilizing Using the DDP1 Expressing Plants and/or Fertilizers Produced Therefrom

Also described herein is methods of using biochar and/or fresh biomass produced from DDP1 expressing plants that have accumulated/sequestered Pi from the soil in which they were grown as a fertilizer. In some embodiments, biochar and/or fresh biomass produced from DDP1 expressing plants that have accumulated/sequestered Pi from the soil in which they were grown is applied to an area in which plants will be grown or are actively growing as a source of phosphorus for growth of the plants.

In some embodiments, biochar and/or fresh biomass is applied 1-10 (e.g., , 2, 3, 4, 5, 6, 7, 8, 9, 10) or more times to an area in which plants will be grown or are growing in a year.

The amount of phosphorous and other nutrient qualities can be measured by any suitable methods and/or techniques. Such methods and/or techniques will be appreciated by one of ordinary skill in the art and/or are described elsewhere herein.

The amount of fresh biomass or biochar applied to any given area, will depend on, for example, the plant to which it is being applied, the starting soil nutrient load, the amount allowed as per nutrient management plan and/or local, state, or federal limit imposed on the particular area, the specific Pi present in the biomass and/or biochar being applied, and the like. Other relevant factors will be appreciated by one of ordinary skill in the art in view of this disclosure.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20° C. and 1 atmosphere.

The Examples herein can at least demonstrate utilization of a yeast phosphatase to artificially remove PP-InsPs in plants. Diadenosine and Diphosphoinositol Polyphosphate Phosphohydrolase (DDP1), a yeast phosphatase, was selected based on its ability to hydrolyze 1-pyrophosphate bonds on PP-InsPs (1PP-InsPs) [18]. As previous approaches utilize Arabidopsis mutants for InsP synthesis enzymes, without being bound by theory, overexpression of an endogenous PP-InsP can uniquely modify the PP-InsPs present in the plant. As demonstrated at least in this Example, DDP1 overexpression in Arabidopsis altered plant PP-InsPs and led to drastic changes in plant physiology and development. These severe physiological and developmental changes are directly dependent on the level of DDP1 expression in stable transgenic and, amount of endogenous Pi present in the media. This approach can uniquely provide a new perspective to which PP-InsPs are important for Pi sensing and signaling as well as the demonstrating that manipulation of PP-InsPs can have significant impacts on plant physiology and development.

Specifically the Examples herein can at least demonstrating that DDP1 overexpression can perturb PP-InsP signaling in vivo, perturbed PP-InsP signaling can upregulate the plant PSR and Pi accumulation, without being bound by theory DDP1 is believed to hydrolyze the PP-InsPs based on the localization of the enzyme in the same compartments of the cell as other PP-InsP synthetic enzymes, and these changes can severely impact plant Pi sensitivity and phenotypes, as these are directly dependent on the level of DDP1 overexpression and endogenous Pi. This can increase in PSR genes in the nonsevere line and minor alterations in the DDP1-H InsP₈ levels demonstrates that minor changes in the PP-InsPs can alter plant Pi sensing. Additional features, advantages, effects, can also be discussed herein.

### SEQUENCES

### Seq Id No: 1

>sp|Q99321|DDP1_YEAST Diphosphoinositol polyphosphate phosphohydrolase DDP1
OS=Saccharomyces cerevisiae (strain ATCC 204508 / S288c) OX=559292 GN-DDP1
PE=1 SV=3 See also GeneBank RefSeq NP_014806.1
MGKTADNHGPVRSETAREGRENQVYSPVT GARLVAGC ICLTPDKKQVLMITSSAH KKRWI
VPKGGVEKDEPNYETTAQRETWEEAGCIGKIVANLGTVEDMRPPKDWNKDIKQFENSRKD
SEVAKHPPRTEFHFYELEIENLLDKFPECHKRHRKLYSYTEAKQNLIDAKRPELLEALNR
SAIIKDDK

### Seq Id No: 2

>Nm_001183582.1 saccharomyces cerevisiae s288c polyphosphatase ddp1 (Ddp1),
Partial mrna
Atgggcaaaaccgcggataatcatggtccagtacgttctgagacagcacgtgaaggaagagaaaaccagg
Tttactcacccgttacaggtgcaagattagttgctggctgcatatgtttaacacccgacaagaagcaagt
Tctcatgattacttcttctgcacacaagaaaagatggattgtccccaaaggtggcgttgagaaagacgag
Cctaattacgagacgactgcccaacgagaaacttgggaggaagctggttgcataggtaaaattgtcgcca
Atttgggtacagttgaagacatgagaccccctaaggactggaacaaagacattaagcaattcgagaactc
Tcgaaaagattcagaagtagcaaagcacccgccaagaaccgaatttcatttttatgaattagagattgaa
Aatctccttgataaatttccggaatgtcacaaaagacatagaaagctatactcttatacagaagctaaac
Aaaacttgatagacgccaagaggcctgaattgttggaggcccttaataggtctgctatcattaaagacga
Caaatag

### Example 1 - DDP1 Overexpression in Arabidopsis Alters Plant Sensitivity to extracellular Pi

Overexpression of the Saccharomyces cerevisiae DDP1 gene (YOR163W) in Arabidopsis Col-0 confers a battery of severe physiological phenotypes. DDP1 OX transgenics are holistically stunted in terms of plant growth and physiology (FIG. 1A). The reproductive capabilities of DDP1 OX are negatively impacted. DDP1 OX Bolts are significantly shorter and thinner compared to WT (FIG. 1B). As DDP1 OX flowers fertilize and siliques, all the flowers towards the bolt base abort before any seeds can be produced. Siliques at the top of each bolt do form siliques with viable seeds however these siliques are smaller than WT siliques, leading to a low recovery of seeds. DDP1 OX rosettes are also severely impacted, growing smaller and accumulating necrotic regions of yellow tissue referred to as leaf lesions (FIG. 1C). DDP1 OX leaf lesions typically appear in DDP1 OX seedlings after about 1.5 to 2 weeks of growth on soil.

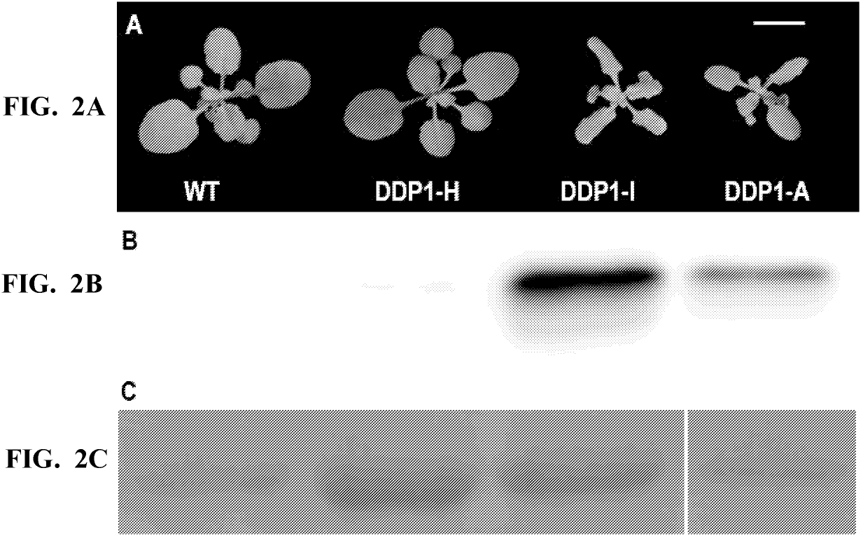

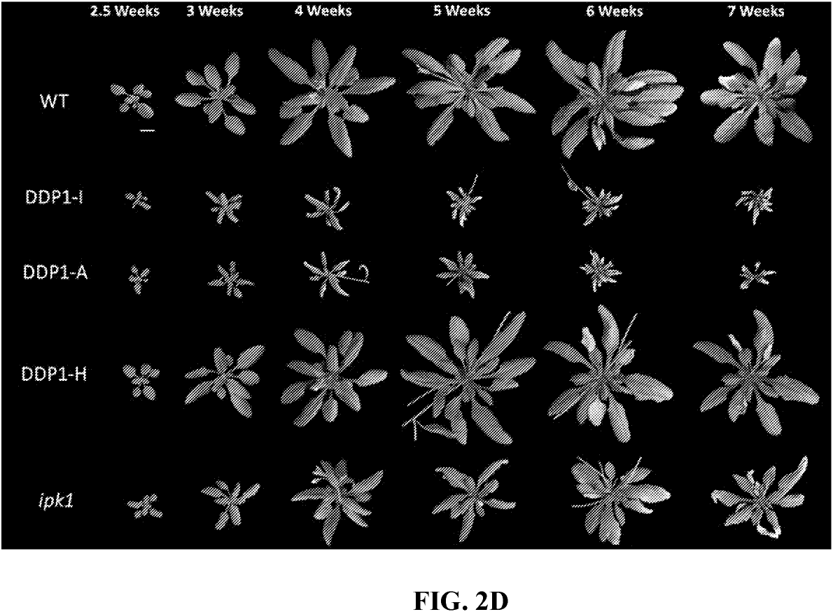

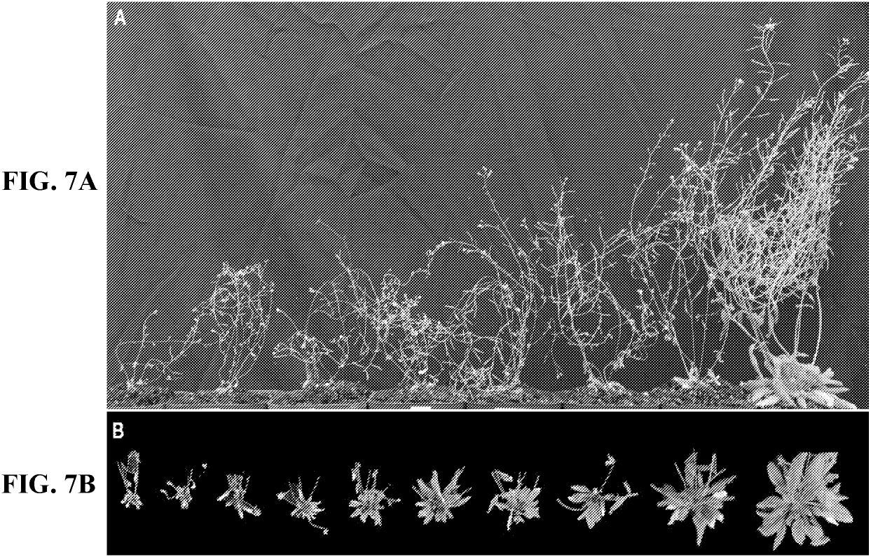

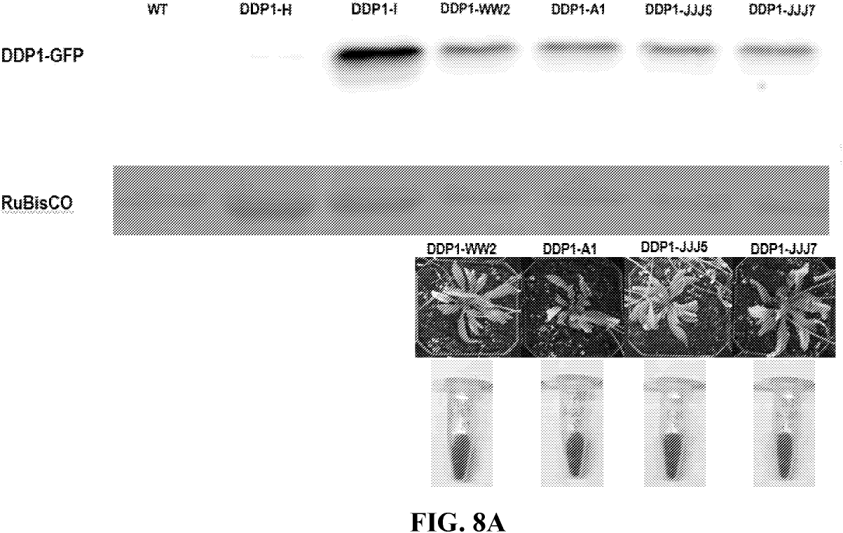

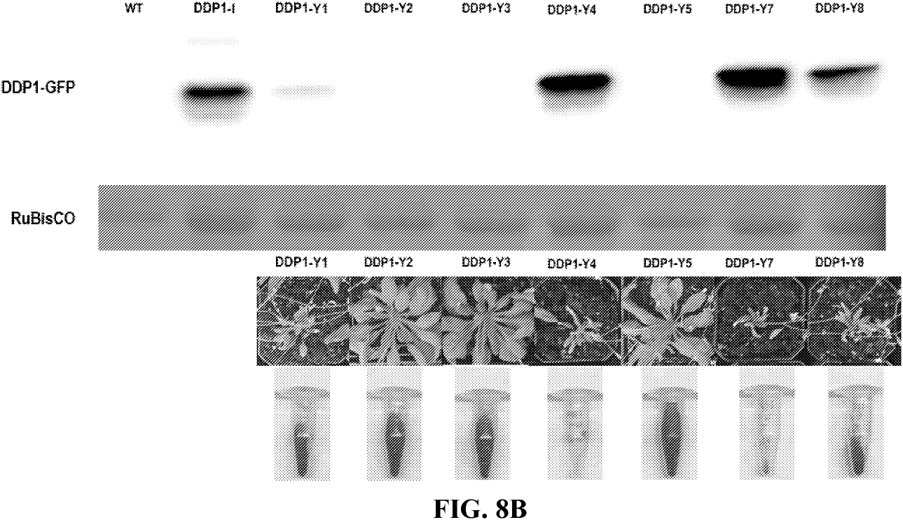

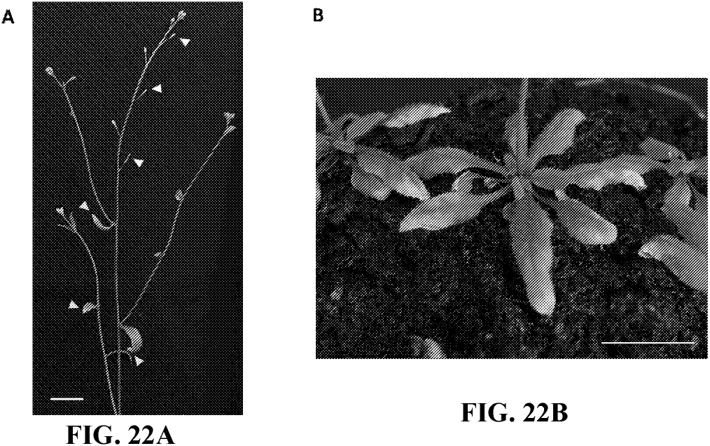

These severe developmental phenotypes manifested in independent transgenic lines as a gradient (FIGS. 7A-7B). Two individual kanamycin screens of DDP1 OX T₀ transgenics were performed, yielding 16 transformants from screen one and 40 from screen two. Of the 16 plants from the first screen, 2 transformants showed the most severe phenotypes, shown in FIGS. 1A-1C. About 8 DDP1 OX transgenics from the second screen of 40 showed highly severe phenotypes. These phenotypes were found to increase in severity as DDP1-GFP protein accumulation increased in the plant tissue, suggesting that the severe phenotypes are dependent of the amount of transgenic DDP1 overexpression to manifest (FIGS. 8A-8B). For this report, 3 lines were selected. Two to represent the severe phenotype DDP1 OX (lines DDP1-I and DDP1-A) and a more WT-like non-severe DDP1 OX line, DDP1-H (FIG. 2A). Severe lines DDP1-I and DDP1-A accumulated higher amounts of DDP1 protein compared to DDP1-H (FIGS. 2B-2C). Additional images of the DDP1 OX phenotype are shown in FIGS. 22A-22B.

To further assess growth of the severe DDP1 OX lines, they were grown in concert with two commonly studied Arabidopsis mutants ipk1 and itpk1. The ipk1 mutant has a disruption in a gene encoding inositol pentakisphosphate 2-kinase, the only enzyme in plants known to synthesize InsP₆ from InsPs [19]. Inositol 1,3,4-trisphosphate ⅚-kinase (ITPK1) synthesizes lower InsP species, InsP₃ and InsP₄, as well as InsP₇ [2,16]. Disruption of these genes in ipk1 and itpk1 mean these mutants are deficient in PP-InsPs and serve as ideal controls to compare with to DDP1 OX transgenics. Severe DDP1 OX lines were found to have significantly smaller rosettes than WT, non-severe DDP1-H and both PP-InsP deficient mutants (FIG. 2D).

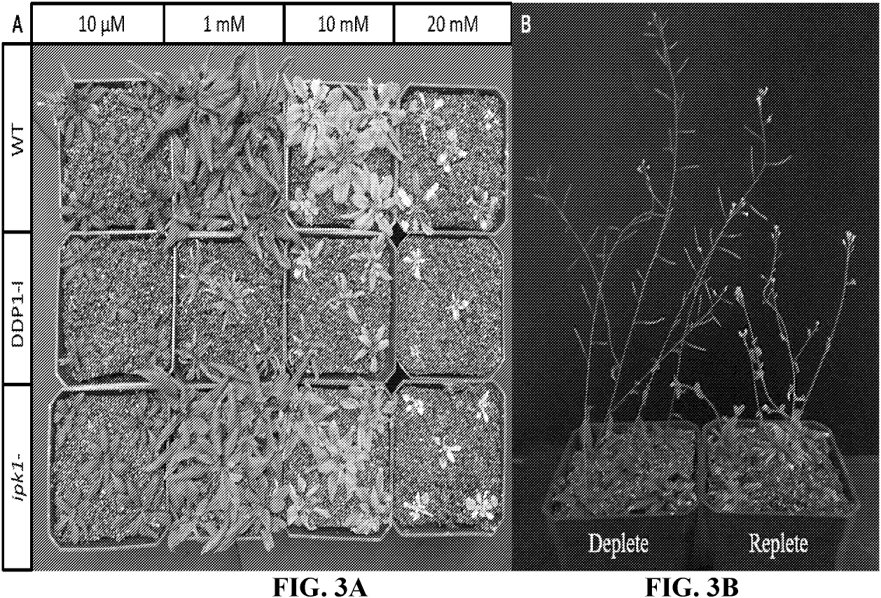

Given the connection between PP-InsPs and Pi sensitivity, it ws determined if these phenotypes were a result of an increased sensitivity to endogenous levels of Pi in the soil. To query this, DDP1 OX transgenics were grown under deplete, replete, and excess Pi conditions on hydroponic media. It was found that DDP1 OX transgenics are highly sensitive to the amount of endogenous Pi (FIG. 3A). Severe phenotypes of DDP1 OX are dependent on the amount of Pi present in the medium. Under depleted Pi, DDP1 OX severe lines did not show a significant difference in rosette diameter growth from WT up until 35 days of growth. DDP1 OX leaves were also darker in color and did not form lesions under deplete Pi. Deplete Pi also rescued the seed abortive phenotype and DDP1 OX plants generated stems and siliques that resembled WT plants (FIG. 3B). This can demonstrate at least DDP1 OX at least alters growth and physiology. The severe phenotypes are directly impacted on 1) level of DDP1 overexpression and 2) the amount of endogenous Pi present.

### Example 2 - DDP1 Overexpression Uniquely Alters PP-InsP Levels

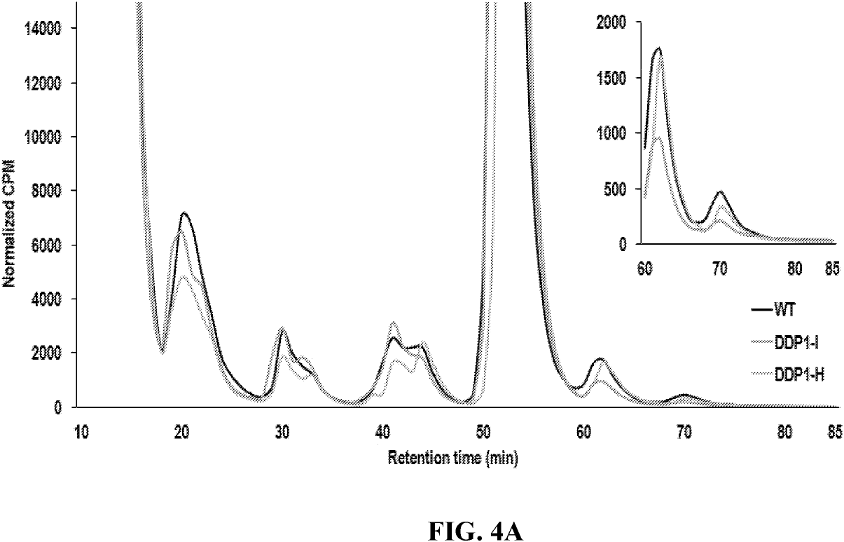

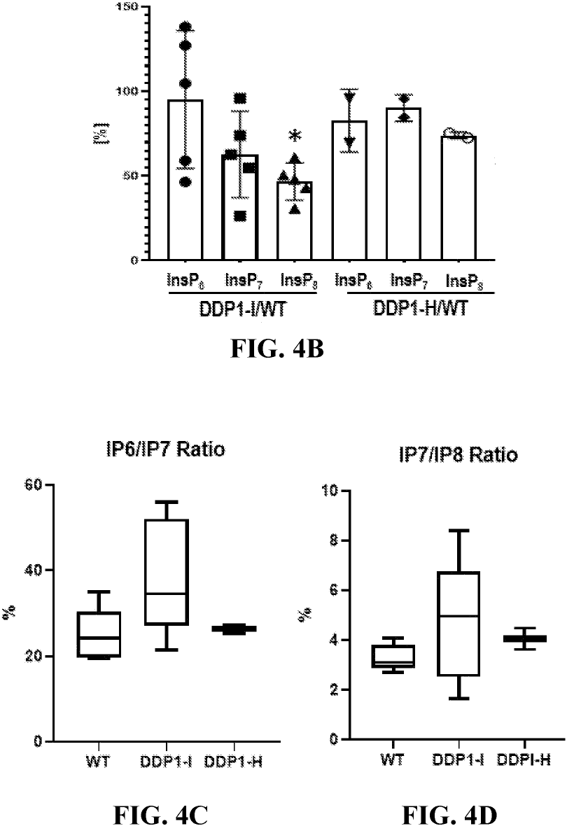

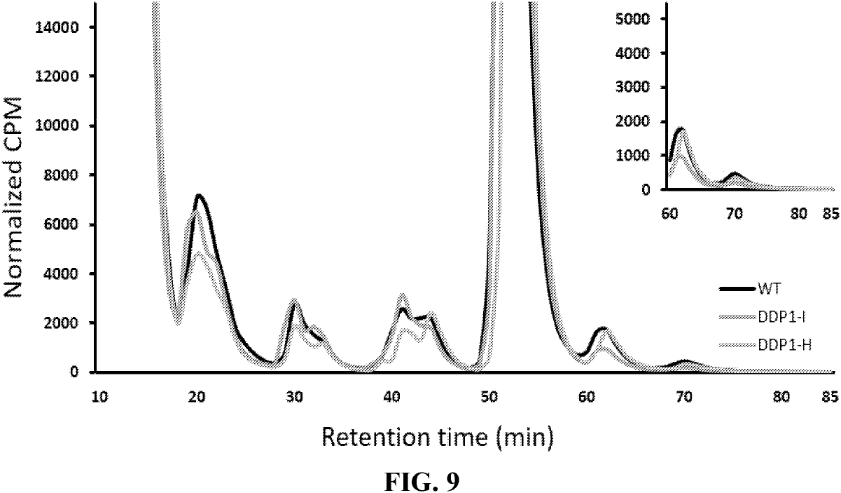

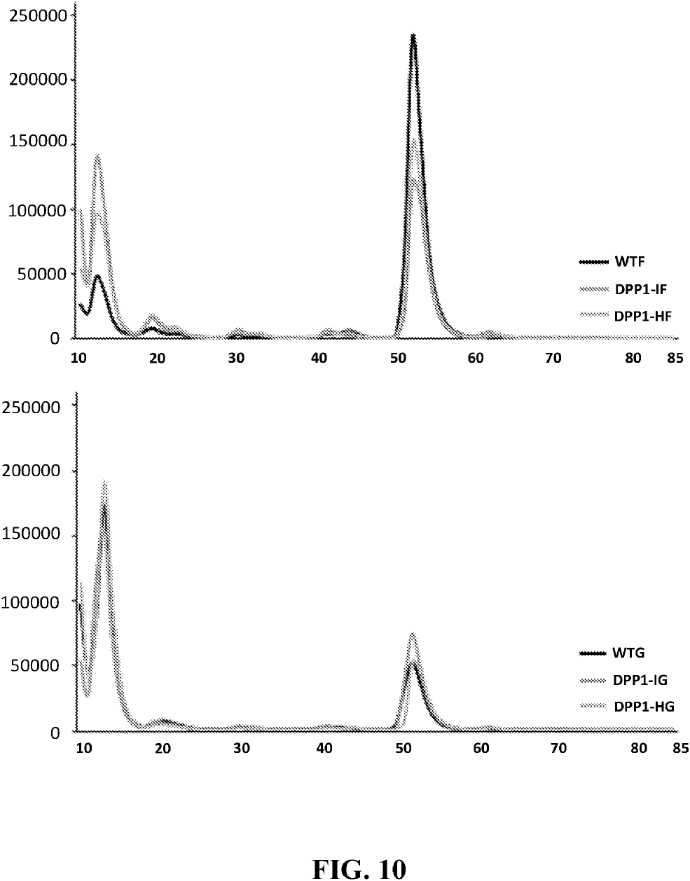

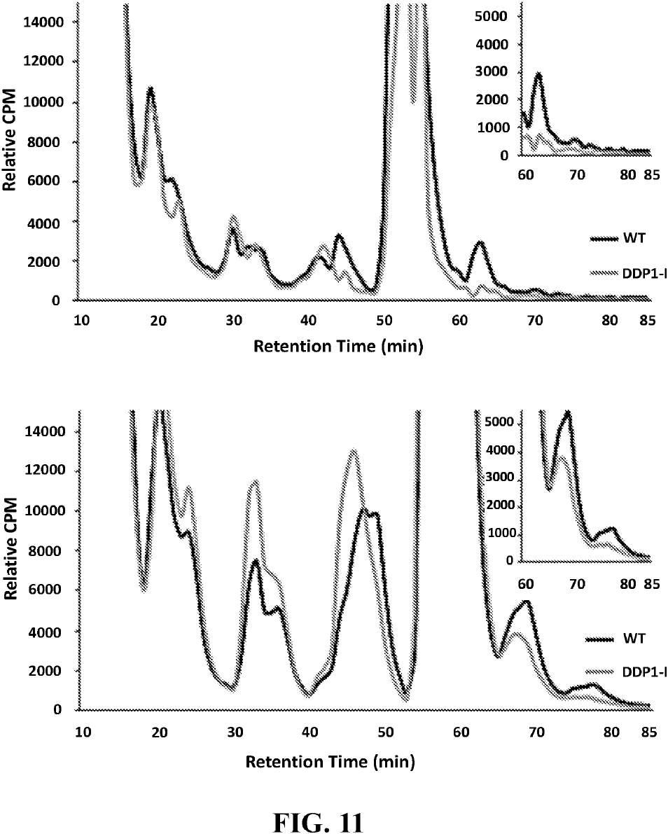

As DDP1 OX severe lines are highly sensitive to endogenous Pi, we queried whether overexpression of DDP1 alters PP-InsPs. It was also gauged whether these phenotypes were dependent on changes in InsP₇ and/or InsPs. To assess PP-InsPs, we compared the InsP and PP-InsP profiles of DDP1-I and DDP1-H to WT through in vivo radiolabeling experiments using myo-[³H] inositol radiolabeling and high-performance liquid chromatography (HPLC) analysis. It was found that the severe DDP1-I line showed a significant in decrease PP-InsPs (FIGS. 4A, 9-11). DDP1-I transgenics had 60-70% InsP₇ and 45-50% InsP₈ of the total WT PP-InsP pool (FIG. 4B). DDP1-H transgenics had more minor decreases in PP-InsPs (92% InsP₇ and 74% InsP₈ of the WT PP-InsP pool). The InsP₆/InsP₇ and InsP₇/InsP₈ ratios were slightly higher in the DDP1-I transgenics than the WT and DDP1-H (FIGS. 4C-4D). InsP₆ and lower InsP levels fluctuated from being higher and lower than WT, suggesting that there are downstream effects. This can demonstrate at least Overexpression of DDP1 uniquely breaks down PP-InsPs from other PP-InsP synthetic mutants. As a result, severe DDP1 OX transgenics are significantly more sensitive to Pi.

### Example 3 - DDP1-GFP Localizes to the Same Cellular Compartments as PP-InsP Synthetic Enzymes

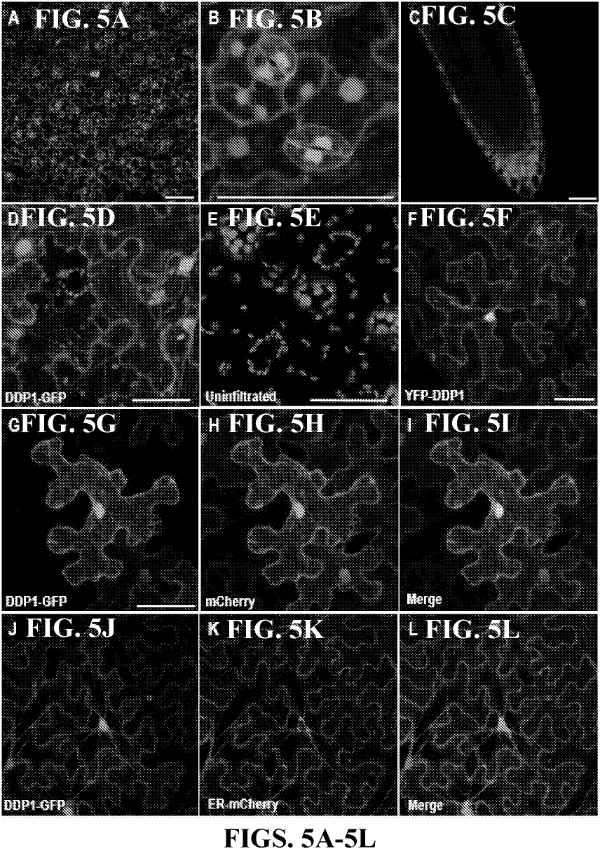

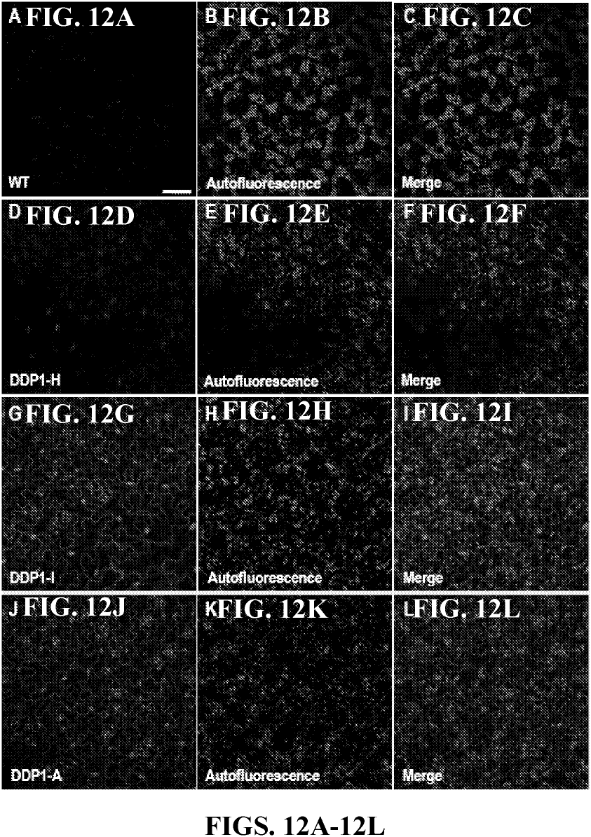

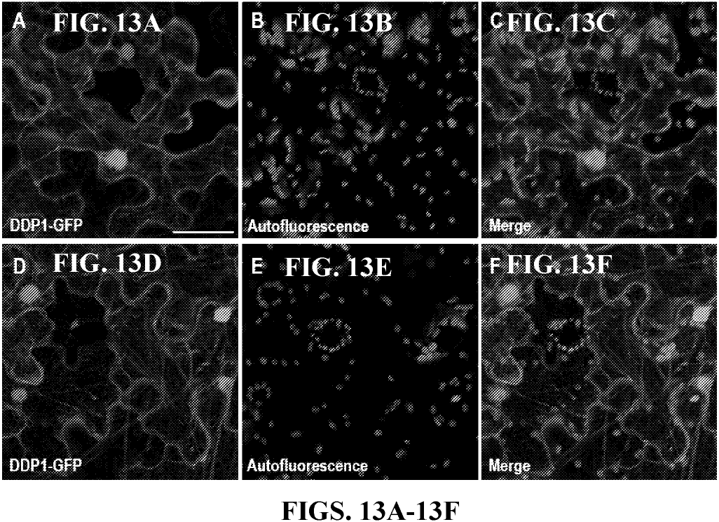

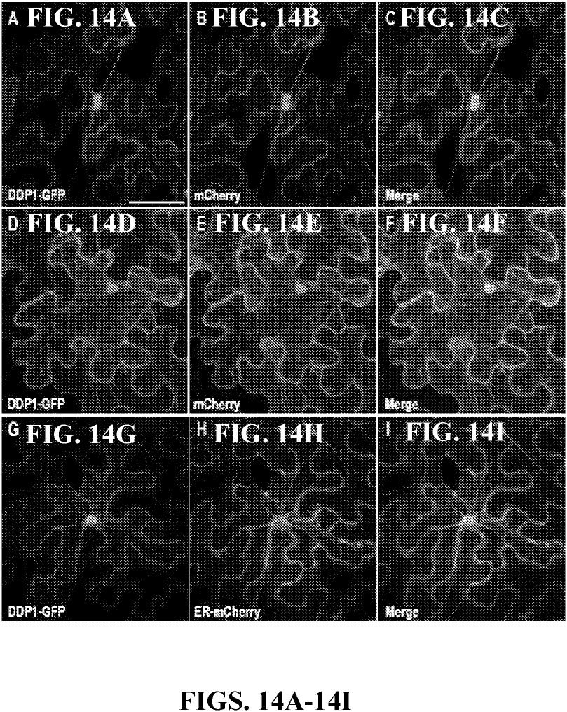

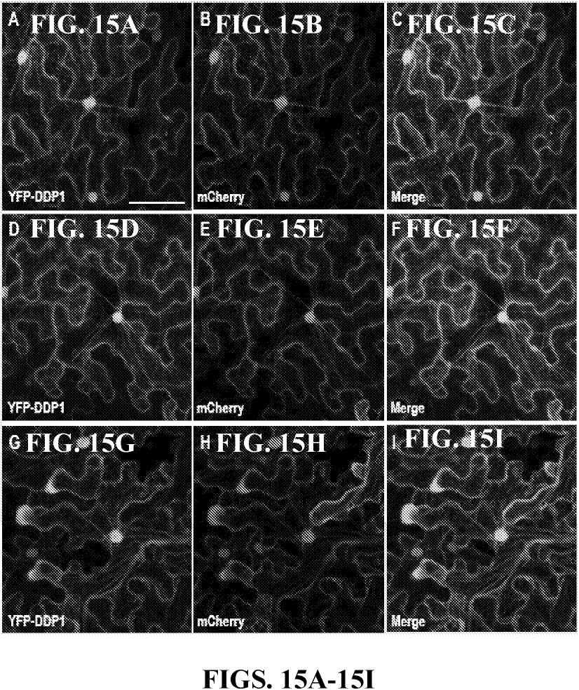

DDP1 subcellular localization was characterized within the plant. As DDP1 is not natively expressed plants, it was especially important to determine the compartments the enzyme localized to compared to the other PP-InsP synthetic enzymes. Nuclei, cytoplasm, and guard cell nuclei. Highly prominent in DDP1-I and DDP1-A lines, showing the severe lines have a higher accumulation of DDP1-GFP than the nonsevere DDP1-H (FIGS. 5A-5C, 12A-12L). DDP1-GFP transiently accumulates in the nucleus and cytoplasm of N. benthamiana leaves (FIGS. 5D, 5G-5L, 13A-13F and 14A-14I). YFP-DDP1 localized to the same compartments as DDP1-GFP, indicating that DDP1 localizes to the cytoplasm and nuclei regardless of whether there is an N- or C-terminal tag (FGIS. 5F and 15A-15I). This can demonstrate at least that DDP1 localizes to cytoplasm and nucleus in both Arabidopsis and N. benthamiana cells as well as Arabidopsis guard cell cytoplasm and nuclei. DDP1-GFP signal was the strongest in DDP1-I and DDP1-A, showing that the severe lines has a greater amount DDP1 overexpression.

### Example 4 - Synthetic Alterations in PP-InsPs Through DDP1 Overexpression Upregulate the PSR

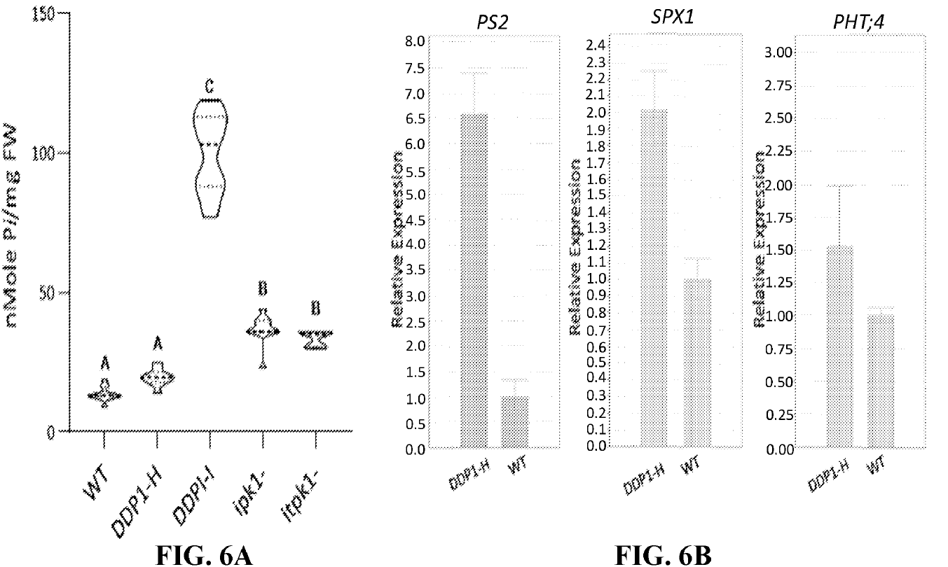

It was assessed whether perturbing PP-InsPs in DDP1 OX led to changes in Pi sensing. Alterations in Pi accumulation and PSR genes in severe DDP1 OX transgenic strongly suggests that DDP1 affects Pi sensing and homeostasis in the transgenics. Severe DDP1 OX transgenics consistently exhibited over a 7-fold increase in accumulation in shoot Pi compared to WT plants (FIG. 6A). This increase in Pi accumulation in DDP1-I was significantly higher than Pi accumulation in both ipk1 and itpk1 mutants. Nonsevere DDP1-H transgenics showed no difference in terms of Pi accumulation from WT plants. The severe phenotypes of the DDP1 OX transgenics and PP-InsP synthetic mutants appear to be related to the amount of Pi accumulation in the plant shoots. Further, increased Pi accumulation is indicative that by overexpressing DDP1 and altering PP-InsPs that transgenics have compromised Pi sensing.

In addition to increased Pi accumulation, DDP1 OX transgenics also exhibited increases in expression of PSR genes. SPX1 expression was examined based on its role in regulating PHR1. Expression of PS2, coding for a pyrophosphate phosphate, and PHT1;4, a Pi transporter, was also measured as these genes are upregulated during Pi starvation and are commonly examined when studying the plant PSR. Without being bound by theory, DDP1 OX transgenics can show a higher increase in PSR genes relative to WT as well as the PP-InsP mutants. Nonsevere DDP1-H seedlings exhibited a 6.5-fold increase in PS2 expression and 2-fold higher expression of SPX1 compared to WT (FIG. 6B).

### Example 5 - Methods for Examples 1-4

### Cloning and Transformation

Cloned DDP1 from Saccharomyces cerevisiae cDNA library into Arabidopsis. DDP1 cloned into gateway vectors pK7FWG2 (C-Terminus GFP) or pH7YWG2,0 (N-Terminus YFP). Both constructs contained a 35S CaMV promoter sequence. DDP1 OX stable transgenics were transformed with pK7FWG2 + DDP1 only. The N-terminus YFP DDP1 construct was used for transient localization experiments only. The DDP1 gene was cloned into both constructs using the Gateway® LR Clonase™ II kit (Invitrogen Corp., Carlsbad, CA). Transformed E. coli colonies were selected using antibiotic selection plates amplified the plasmid and were sequenced. Agrobacterium (strain GV3101) was transformed with these constructs and were used for transient Nicotiana benthamiana experiments or to transform Arabidopsis thaliana (ecotype Col-0) plants.

### Plant Growth Conditions

ipk1 and itpk1 T-DNA mutants were obtained from the Arabidopsis Biological Resource Center (ABRC, Columbus, OH, USA). All mutants, transgenics, and WT Arabidopsis lines were identically grown in concert on soil under long day conditions (16 hours light/8 hours dark, 55% humidity, day/night temperature 23/21° C. and 120 µE light intensity). Seedlings grown under sterile conditions were first sterilized using 100% ethanol for one minute, transferred to a 30% (v/v) Clorox solution for 5 minutes and washed five times with ddH₂O. Seeds were placed in 0.5X MS media + 0.2% agar and stratified for 3 days at 4° C. Seeds were either transferred to 0.5X MS solid media plates or multi-well plates containing semi-solid media (0.2% agar). For the hydroponics experiment, stratified seeds were added to a solution containing 0.01% agarose and placed on top of pots containing washed vermiculite. All pots were placed in trays containing 2 liters of 0.25X MS media and varying Pi (deplete (10 µM), replete (1 mM), excess (10 and 20 mM) KH₂PO₄). The rosette diameters were measured in all plants grown on soil and hydroponics; all data was analyzed using JMP Pro and GraphPad Prism.

### Protein Blots of GFP-Fusion Proteins

Conditions were previously reported [20]. Leaf tissue from 4-week old soil grown plants was ground up in liquid nitrogen, homogenized and separated from cell debris. SDS-bromophenol blue loading dye was added to the extracted proteins and boiled for 5 minutes at 85° C. After a subsequent centrifugation, the SN was loaded onto a polyacrylamide gel; equal amounts of protein were added from each sample. For western blotting, a 1: 5000 dilution of anti-GFP antibody (Invitrogen Molecular Probes, Eugene, OR) and a 1: 2000 dilution of secondary goat anti-rabbit horseradish peroxidase antibody (Bio-Rad Laboratories, Hercules, CA) were used to detect GFP.

### InsP and PP-InsP Seedling Profiles

WT and DDP1 OX transgenics were grown in semi-solid 0.5X MS media and 0.2% agar for two weeks, 30 µE light intensity. 26 seedlings of each genotype were transferred to Eppendorf tubes containing 300 µL of 0.5X MS media and 0.2% agar and 100 µCi of [³H] myo-inositol (20 Ci/mmol; American Radiolabeled Chemicals (ARC), St. Louis, MO, USA) for 4 days. InsPs were extracted from seedlings by pulverizing tissue in extraction buffer (1 M Perchloric acid (HClO₄), 25 mM EDTA, and 10 mg/ml InsP₆) and vortexing with glass beads. The pH of the solution was neutralized to pH 6-8 using a neutralization buffer (250 mM EDTA and 1 M K₂CO₃). Samples were dried to a volume of 80-100 µL. Negatively charged species were analyzed HPLC using a 125 × 4.6 mm Partisphere SAX-column (Sigma-Aldrich, St. Louis, MO, USA) and separated using an elution gradient [21]. Radioactivity in all collected fractions was quantified using a scintillation counter.

### Subcellular Localization and Imaging

All Arabidopsis and N. benthiamiana cells were imaged using a Zeiss LSM 880 microscope (Carl Zeiss) and examined using a 40× C-Apochromat water immersion lens. N. benthamiana plant leaves were infiltrated with transformed Agrobacterium tumefaciens, as previously described (Kapila et al., 1997). Agrobacterium cells were grown overnight, pelleted, and resuspended in MMA (10 mM MES, 1× MS, 200 µM acetosyringone) solution at an OD₆₀₀ of 1.0. After a 2-4 hour incubation period, N. benthamiana leaves were infiltrated with the Agrobacterium MMA cultures. Leaf sections were imaged after 24, 48, and 72-hours post infiltration. GFP was excited using a 488-nm argon laser and its fluorescence was detected at 500- to 550-nm and YFP was excited using a 488-nm argon laser and its fluorescence was detected at 500- to 550-nm. GFP- and YFP-tagged proteins were colocalized with a set of mCherry tagged organelle markers, which were mCherry was imaged using excitation with a 594-nm laser and fluorescence was detected at 600- 650-nm. Chlorophyll autofluorescence was collected using a 594-nm laser and emission above 650 nm was collected.

### Pi Accumulation Assays

Pi was extracted from 100 mg of 4-week old soil grown Arabidopsis leaf tissue. Samples were pulverized in liquid nitrogen. A 1:10 ratio of 1% acetic acid was added to each sample, vortexed and incubated on ice, and centrifuged. 50 µL of the supernatant and 1 mL of working reagent (Ferrous Sulfate + Ammonium Molybdate solution) in the dark. Samples were vortexed and exposed to light. After one hour, samples were placed in quartz cuvettes and the A660 was read using a cuvette reader. A set of Pi standards was made for each replicate experiment to generate a standard curve in which all samples could be compared.

### Quantitative Real-Time PCR

RNA was extracted from 2 week-old seedlings grown on 0.5X MS media + 0.2% agar well plated using the Plant RNeasy kit (Qiagen). During this process, samples were DNAse treated with DNA-free Turbo (Invitrogen). RNA reverse transcription process utilized 2 µg of total RNA Multiscribe Transcriptase (Applied Biosystems). For quantitative PCR, SYBRgreen, specific primers for AtPS2, AtPHT1;4, and AtSPX1 as well as 20 ng of cDNA were used as previously described [10]. Relative expression was calculated using the ΔΔCT method.

### Example 6 - DDP1 Overexpressing Thlaspi Arvense (Pennycress)

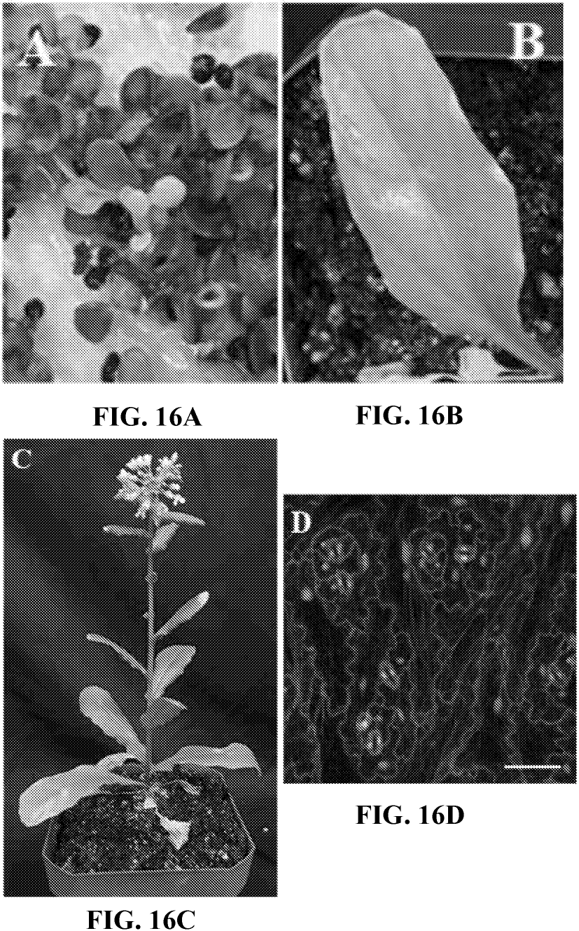

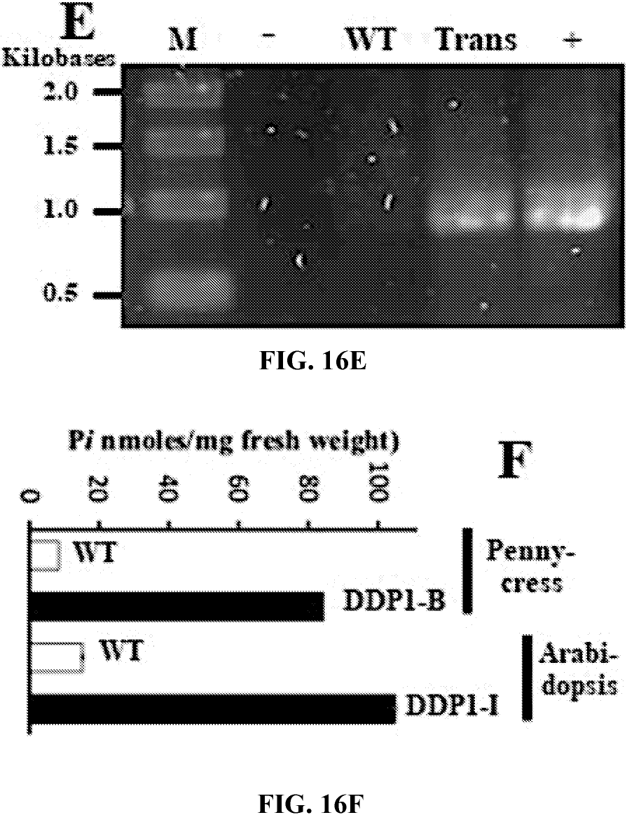

As shown in e.g., prior Working Examples, DDP1 was expressed in Arabidopsis. This Example demonstrates that DDP1 is capable of being overexpressed in other plants and that its overexpression leads to accumulation of Pi as well, which can demonstrate that such an approach is suitable for a wide variety of plant species. Pennycress is generally used by farmers as a cover crop to protect soils from nutrient leaching and erosion. FIGS. 16A-16F show phenotypes of Pennycress DDP1 overexpressors (PcDDP1 OX).

This example can at least demonstrate that Pennycress plants can be stably transformed and overexpress DDP1 and hyperaccumulate Pi FIGS. 16A-16F. Pennycress DDP1 OX transgenics (PcDDP1 OX) exhibited similar physiological phenotypes to AtDDP1 OX transgenics, one being accumulation of leaf lesions after a few weeks of growth (FIGS. 16B-16C). PcDDP1 OX transgenics overexpress DDP1 with an N-terminal yellow fluorescent protein (YFP) tag. YFP-DDP1 protein accumulates in the epidermal and guard cell nuclei and the cytoplasm, which is equivalent in AtDDP1 OX cells (FIG. 16D). Most prominently, PcDDP1 OX exhibit a similar fold increase in Pi accumulation when compared to WT (FIG. 16F). These data suggest that DDP1 is localizing to the same compartments in Arabidopsis and pennycress to hydrolyze PP-InsPs. Unique hydrolysis of PP-InsPs leads to alterations in physiology, such as leaf lesions, as well as hyperaccumulation of Pi in above ground plant tissue.

### Example 7 - DDP1 Overexpression Reduces Seedling PP-InsP Levels

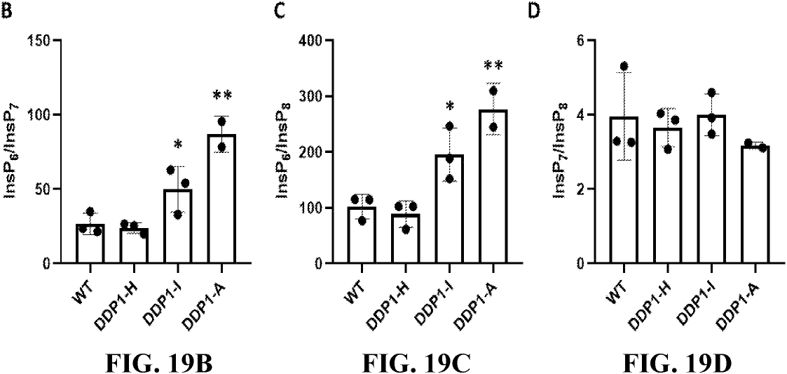

To determine whether DDP1 overexpression alters PP-InsP profiles in planta, the InsP and PP-InsP profiles of all three DDP1 OX lines was compared to WT through in vivo radiolabeling experiments using myo-[³H] inositol radiolabeling and high-performance liquid chromatography (HPLC) analysis. DDP1-I and DDP1-A showed a significant in decrease PP-InsPs (FIG. 19A). DDP1-I transgenics had 41-62% InsP₇ of the total WT InsP₇ pool and DDP1-A transgenics had slightly lower InsP₇ than DDP1-I with 32-33% of the total WT InsP₇ pool. Both severe lines showed equivalent InsPs pools to WT, with DDP1-I at 44-55% and DDP1-A at 55% of the total WT InsPs pool. DDP1-H transgenics had little to no changes in PP-InsPs compared to WT; in two replicates, DDP1-H showed 84-95% InsP7 and 71-78% InsP₈ of the WT PP-InsP pool and in a separate replicate 125% InsP₇ and 216% InsPs compared to WT.

The InsP₆/InsP₇ and InsP₆/InsP₈ ratios were significantly higher in the DDP1-I and DDP1-A transgenics than the WT and DDP1-H (FIGS. 19B-19C). When compared to each other, DDP1-A had a larger decrease in InsP₆/InsP₇ and InsP₆/InsP₈ ratios compared to DDP1-I however when comparing the total pool of PP-InsPs of the severe transgenics to WT, the decreases in PP-InsPs are comparable and repeatable in both lines. There was no significant difference in InsP₇/InsP₈ ratio between WT and the DDP1 OX lines, suggesting (FIG. 19D).

### Example 8 - DDP1 Overexpressing Plants Hyperaccumulates Pi Throughout its Life

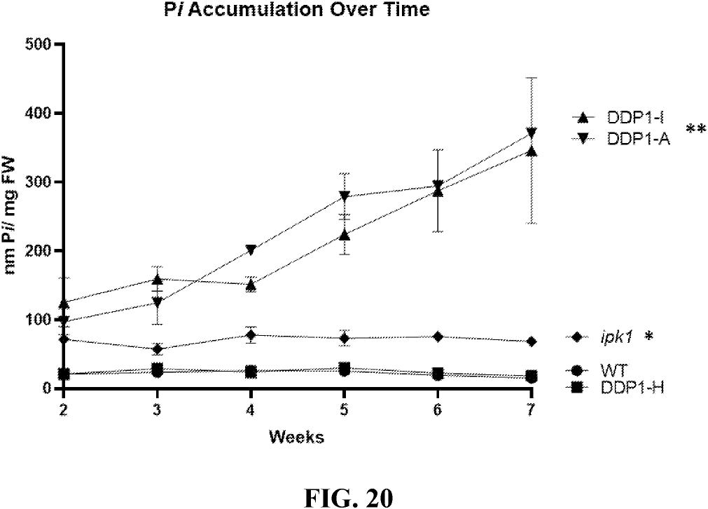

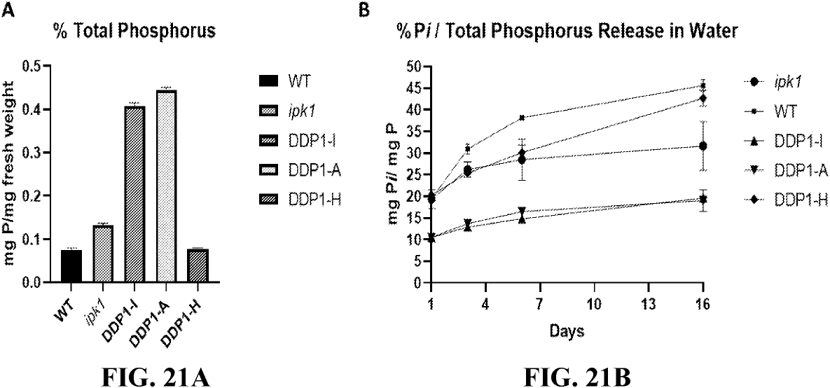

DDP1 overexpressing lines can hyperaccumulate significantly more Pi over the entire course of their life span (FIG. 20), which can result in in a Pi toxicity. It will be appreciated that this toxicity may not influence productivity for certain applications.

### Example 9 - Slow-Release Fertilizer From DDP1 Overexpressing Plants

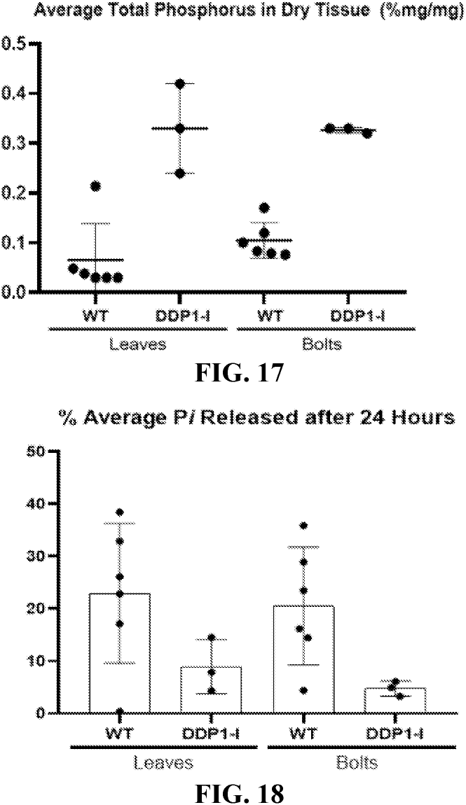

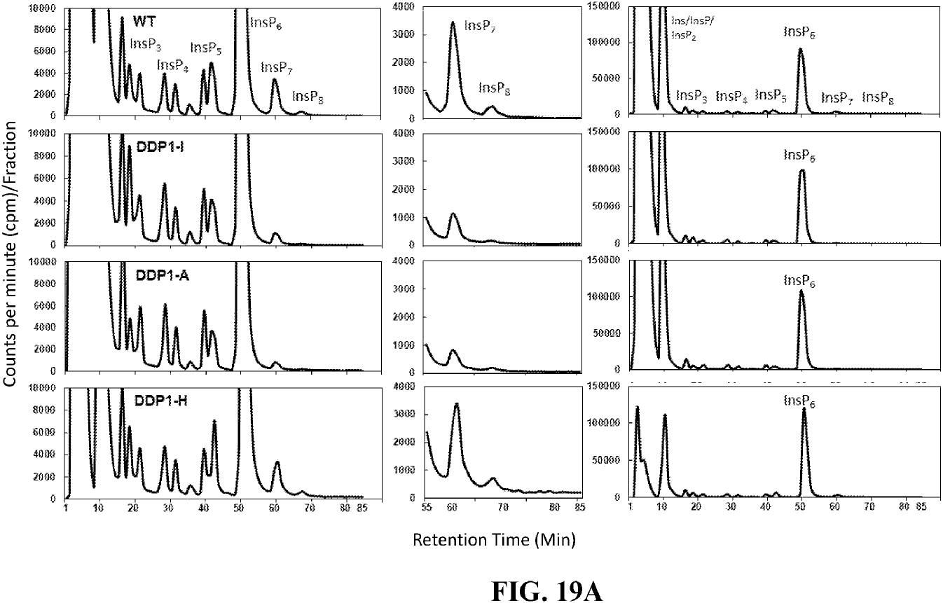

Without being bound by theory it is believed that the sequestered Pi in the DDP1 overexpressing plants can be distributed to Pi-deficient agricultural land. To achieve this, the DDP1 overexpressing plants or components thereof can be processed into a fertilizer that can be applied to soils. This Example can at least demonstrate the carbonization of the plant material from DDP1 overexpressing plants into biochar. The carbonization process ensures the biochar retains most of the macronutrients and micronutrients collected. The data presented herein revealed that AtDDP1 OX leaves produced biochar that was approximately 6 times richer in phosphorus than WT Arabidopsis plants (FIG. 17). As plant-absorbed phosphorus is highly mobile, the mobility of phosphorus in water was further investigated to evaluate it as a slow-releasing fertilizer. Less than 10% of AtDDP1 OX biochar Pi was soluble while 20-30% of phosphorus in WT biochar was soluble after 24 hours (FIG. 18). Without being bound by theory, the decrease in soluble phosphorus was attributed to a significant increase in multivalent cations, such as Al, Ca, Mg, and Fe, in the DDP1 OX. This suggests that the sequestered P is bound to these metal compounds and won’t be quickly biologically available, which demonstrates that it is capable of utilization as a long-term source of P for biota.

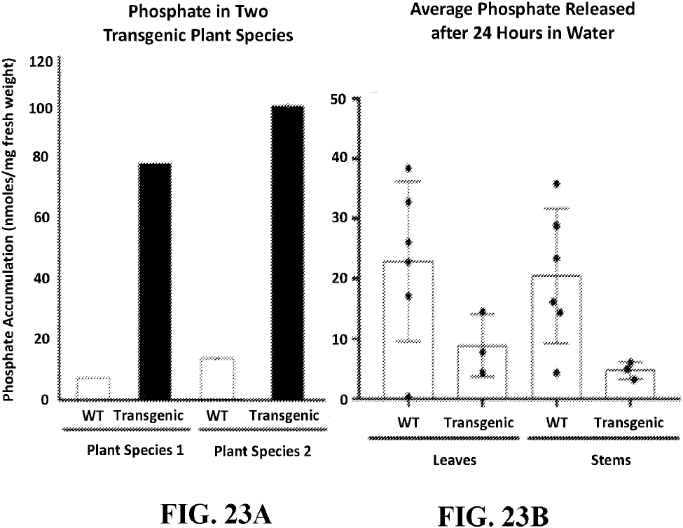

FIGS. 23A-23B show inorganic phosphate accumulation in and release from processed plants from two species engineered to express a heterologous DDP1.

Overall, these data support that Pi sequestered from soil grown DDP1-overexpressing plants can be recycled in an agriculturally desirable form. Cumulatively the Working Examples herein demonstrate that plants expressing a heterologous DDP1 provide a unique platform to sequester Pi from the soil as well as in turn provide a source for a fertilizer to recycle the Pi that was sequestered and a solution to both reducing Pi pollution and fertilizer consumption while redistributing nutrients to Pi-deficient agricultural land.

The figures herein are for illustrative purposes only and are not necessarily drawn to scale.

### REFERENCES FOR EXAMPLES

1. Kanno, S.; Cuyas, L.; Javot, H.; Bligny, R.; Gout, E.; Dartevelle, T.; Hanchi, M.; Nakanishi, T.M.; Thibaud, M.-C.; Nussaume, L. Performance and Limitations of Phosphate Quantification: Guidelines for Plant Biologists. Plant Cell Physiol 2016, 57, 690-706, doi:10.1093/pcp/pcv208.

2. Kuo, H.-F.; Hsu, Y.-Y.; Lin, W.-C.; Chen, K.-Y.; Munnik, T.; Brearley, C. Arabidopsis inositol phosphate kinases IPK1 and ITPK1 constitute a metabolic pathway in maintaining phosphate homeostasis. The Plant Journal 2018, 95, 613-630.

3. Sattari, S.Z.; Bouwman, A.F.; Giller, K.E.; van Ittersum, M.K. Residual soil phosphorus as the missing piece in the global phosphorus crisis puzzle. Proc Natl Acad Sci USA 2012, 109, 6348-6353, doi:10.1073/pnas.1113675109.

4. Song, L.; Liu, D. Ethylene and plant responses to phosphate deficiency. Front. Plant Sci. 2015, 6, doi:10.3389/fpls.2015.00796.

5. Rouached, H.; Arpat, A.B.; Poirier, Y. Regulation of Phosphate Starvation Responses in Plants: Signaling Players and Cross-Talks. Molecular Plant 2010, 3, 288-299, doi:10.1093/mp/ssp120.

6. Chien, P.-S.; Chiang, C.-P.; Leong, S.J.; Chiou, T.-J. Sensing and Signaling of Phosphate Starvation - from Local to Long Distance. Plant Cell Physiol. 2018, 59, 1714-1722, doi:10.1093/pcp/pcy 148.

7. Williams, S.P.; Gillaspy, G.E.; Perera, I.Y. Biosynthesis and possible functions of inositol pyrophosphates in plants. Front Plant Sci 2015, 6, doi:10.3389/fpls.2015.00067.

8. Freed, C.; Adepoju, O.; Gillaspy, G. Can Inositol Pyrophosphates Inform Strategies for Developing Low Phytate Crops? Plants 2020, 9, 115, doi:10.3390/plants9010115.

9. Nagy, R.; Grob, H.; Weder, B.; Green, P.; Klein, M.; Frelet-Barrand, A.; Schjoerring, J.K.; Brearley, C.; Martinoia, E. The Arabidopsis ATP-binding cassette protein AtMRP5/AtABCC5 is a high affinity inositol hexakisphosphate transporter involved in guard cell signaling and phytate storage. J. Biol. Chem. 2009, 284, 33614-33622, doi:10.1074/jbc.M109.030247.

10. Desai, M.; Rangarajan, P.; Donahue, J.L.; Williams, S.P.; Land, E.S.; Mandal, M.K.; Phillippy, B.Q.; Perera, I.Y.; Raboy, V.; Gillaspy, G.E. Two inositol hexakisphosphate kinases drive inositol pyrophosphate synthesis in plants. The Plant Journal 2014, 80, 642-653, doi:10.1111/tpj.12669.

11. Raboy, V.; Young, K.A.; Dorsch, J.A.; Cook, A. Genetics and breeding of seed phosphorus and phytic acid. Journal of Plant Physiology 2001, 158, 489-497, doi:10.1078/0176-1617-00361.

12. Tan, X.; Calderon-Villalobos, L.I.A.; Sharon, M.; Zheng, C.; Robinson, C.V.; Estelle, M.; Zheng, N. Mechanism of auxin perception by the TIR1 ubiquitin ligase. Nature 2007, 446, 640-645, doi:10.1038/nature05731.

13. Laha, D.; Johnen, P.; Azevedo, C.; Dynowski, M.; Weiß, M.; Capolicchio, S.; Mao, H.; Iven, T.; Steenbergen, M.; Freyer, M.; et al. VIH2 Regulates the Synthesis of Inositol Pyrophosphate InsP8 and Jasmonate-Dependent Defenses in Arabidopsis. The Plant Cell 2015, 27, 1082-1097, doi:10.1105/tpc.114.135160.

14. Adepoju, O.; Williams, S.P.; Craige, B.; Cridland, C.A.; Sharpe, A.K.; Brown, A.M.; Land, E.; Perera, I.Y.; Mena, D.; Sobrado, P.; et al. Inositol Trisphosphate Kinase and Diphosphoinositol Pentakisphosphate Kinase Enzymes Constitute the Inositol Pyrophosphate Synthesis Pathway in Plants. bioRxiv 2019, 724914, doi:10.1101/724914.

15. Zhu, J.; Lau, K.; Harmel, R.K.; Puschmann, R.; Broger, L.; Dutta, A.K.; Jessen, H.J.; Hothorn, L.A.; Fiedler, D.; Hothorn, M. Two bifunctional inositol pyrophosphate kinases/phosphatases control plant phosphate homeostasis. bioRxiv 2018, 467076, doi:10.1101/467076.

16. Laha, D.; Parvin, N.; Hofer, A.; Giehl, R.F.H.; Femandez-Rebollo, N.; von Wiren, N.; Saiardi, A.; Jessen, H.J.; Schaaf, G. Arabidopsis ITPK1 and ITPK2 Have an Evolutionarily Conserved Phytic Acid Kinase Activity. ACS Chem. Biol. 2019, 14, 2127-2133, doi:10.1021/acschembio.9b00423.

17. Dong, J.; Ma, G.; Sui, L.; Wei, M.; Satheesh, V.; Zhang, R.; Ge, S.; Li, J.; Zhang, T.-E.; Wittwer, C.; et al. Inositol Pyrophosphate InsP8 Acts as an Intracellular Phosphate Signal in Arabidopsis. MolPlant 2019, 12, 1463-1473, doi:10.1016/j.molp.2019.08.002.

18. Kilari, R.S.; Weaver, J.D.; Shears, S.B.; Safrany, S.T. Understanding inositol pyrophosphate metabolism and function: kinetic characterization of the DIPPs. FEBS Lett 2013, 587, doi:10.1016/j.febslet.2013.08.035.

19. Stevenson-Paulik, J.; Bastidas, R.; Chiou, S.-T.; Frye, R.; York, J. Generation of phytate-free seeds in Arabidopsis through disruption of inositol polyphosphate kinases. Proceedings of the National Academy of Sciences of the United States of America 2005, 102, 12612-12617.

20. Burnette, R.N.; Gunesekera, B.M.; Gillaspy, G.E. An Arabidopsis inositol 5-phosphatase gain-of-function alters abscisic acid signaling. Plant Physiol. 2003, 132, 1011-1019, doi:10.1104/pp.102.019000.

21. Azevedo, C.; Saiardi, A. Extraction and analysis of soluble inositol polyphosphates from yeast. Nature Protocols 2006, 1, 2416-2422, doi:10.1038/nprot.2006.337.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered plant cell comprising:
a heterologous Diadenosine and Diphosphoinositol Polyphosphate Phosphohydrolase (DDP1) polypeptide, a heterologous DDP1 encoding polynucleotide, a vector or vector system comprising a heterologous DDP1 encoding polynucleotide, or a combination thereof.

2. The engineered plant cell of claim 1, wherein the plant cell expresses the heterologous DDP1 polypeptide, the heterologous DDP1 encoding polynucleotide, the vector or vector system comprising the heterologous DDP1 encoding polynucleotide, or a combination thereof.

3. The engineered plant cell of any of claims 1-2, wherein the heterologous DDP1 is a fungi DDP1 or a mammalian DDP1.

4. The engineered plant cell of claim 4, wherein the fungi DDP1 is a yeast DDP1.

5. The engineered plant cell of claim 5, wherein the yeast DDP1 is a DDP1 from the genus Saccharomyces, Candida, Zygosaccharomyces, Kluyveromyces, Babjeviella, Kazachstania, Torulaspora, Tetrapisispora, Lachancea, Naumovozyma, and related strains.

6. The engineered plant cell of any of claims 1-5, wherein the DDP1 is a Saccharomyces cerevisiae DDP1.

7. The engineered plant cell of any one of claims 1-6, wherein the DDP1 polypeptide is about 50-100% identical to SEQ ID NO: 1.

8. The engineered plant cell of any one of claims 1-7, wherein the DDP1 encoding polynucleotide is about 50-100% identical to SEQ ID NO: 2.

9. An engineered plant comprising:
one or more cells as in any one of claims 1-8.

10. The engineered plant of claim 9, wherein the engineered plant is an engineered monocotyledonous plant or an engineered dicotyledonous plant.

11. The engineered plant of claim 10, wherein the engineered dicotyledonous plant belongs to the order Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violates, Salicales, Capparales, Ericales, Diapensales, Ebenales, Brassicales, Primulales, Rosales, Fabales, Podostemales, Haloragales, Myrtales, Comales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, or Asterales.

12. The engineered plant of claim 10, wherein the engineered monocotyledonous plant belongs to the order Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, Orchid ale, Pinales, Ginkogoales, Cycadales, Araucariales, Cupressales or Gnetales.

13. The engineered plant of any of claims 9-13, wherein the engineered plant is a species of Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Thlaspi, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, or Pseudotsuga.

14. The engineered plant of any one of claims 9-13, wherein the plant is a grain crop, a fruit crop, forage crops, a root vegetable crop plant, a leafy vegetable crop plant; a flowering crop plant, a conifers or pine tree; a plant used in phytoremediation; an oil crop plant), a ground cover, a turfgrass, or a plant typically used for experimental purposes.

15. The engineered plant of any one of claims 9-14, wherein the engineered plant is an angiosperm or a gymnosperm plant.

16. The engineered plant of any one of claims 9-16, wherein the engineered plant is acacia, alfalfa, amaranth, apple, apricot, artichoke, ash tree, asparagus, avocado, banana, barley, beans, beet, birch, beech, blackberry, blueberry, broccoli, Brussel’s sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pennycress, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, or zucchini.

17. The engineered plant of any of claims 9-16, wherein the engineered plant is a turfgrass.

18. The engineered plant of any of claims 9-17, wherein the engineered plant is an algae.

19. The engineered plant of claim 18, wherein the engineered plant is an algae from the phyla Rhodophyta, Chlorophyta, Phaeophyta, Bacillariophyta, Eustigmatophyta, a dinoflagellates, or the prokaryotic phylum Cyanobacteria.

20. The engineered plant of any of claims 18-19, wherein the engineered algae the species of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, or Trichodesmium.

21. The engineered plant of claim 9, wherein the engineered plant is a fern, moss, or liverwort.

22. The engineered cell of any one of claims 1-8 or the engineered plant of any of claims 9-21, wherein the engineered cell, the engineered plant, or both comprise a DDP1 encoding polynucleotide stably integrated into the genome of the engineered cell.

23. The engineered cell of any of claims 1-8 or the engineered plant of any of claims 9-21, wherein the engineered cell, the engineered plant, or both comprise a DDP1 encoding polynucleotide that is transiently expressed in the engineered cell, engineered plant, or both.

24. The engineered plant of any of claims 9-23, wherein the engineered plant or cell thereof has one or more modulated observable traits as compared to an unmodified plant.

25. The engineered plant of any of claims 9-24, wherein the engineered plant has increased growth and/or or performance in at least one economically important trait, optionally wherein the trait comprises improved Pi utilization, accumulation, and/or storage, growth, fruit yield, flower yield, hardiness, stress tolerance, or any combination thereof.

26. A method comprising: modifying a plant cell such that the plant cell comprises a heterologous Diadenosine and Diphosphoinositol Polyphosphate Phosphohydrolase (DDP1) polypeptide, a heterologous DDP1 encoding polynucleotide, a vector or vector system comprising a heterologous DDP1 encoding polynucleotide, or any combination thereof.

27. The method of claim 26, wherein modifying comprises delivering a polynucleotide having a sequence that is about 50-100% identical to SEQ ID NO: 2 to the cell, delivering a polypeptide having a sequence that is about 50-100% identical to SEQ ID NO: 1 to the cell, or both.

28. A method comprising growing, propagating, harvesting, and/or cultivating a plant as in any one of claims 9-25.

29. A method of removing Pi from a soil or water, comprising: growing, propagating, harvesting, and/or cultivating a plant as in any one of claims 9-25 in the soil or water.

30. A method of producing biochar, the method comprising:
carbonizing biomass from a plant as in any one of claims 9-25 by a suitable process to form the biochar.

31. A biochar and/or a fertilizer produced from a DDP1 overexpressing plant.

32. The biochar of claim 31, wherein the DDP1 overexpressing plant is as in any one of claims 9-25.

33. The biochar of any one of claims 31-32, wherein the biochar releases phosphorus at a rate 1-100 times slower as compared to biochar made from a wild-type of non-DDP1 expressing control plant.

34. A method of applying nutrients and/or a fertilizer to a soil, the method comprising:
applying an amount of biomass from a DDP1 overexpressing plant and/or a biochar produced from a DDP1 overexpressing plant to the soil.

35. The method of claim 34, wherein the biomass is fresh biomass.

36. The method of any one of claims 34-35, wherein the DDP1 over expressing plant is as in any one of claims 9-25.
